(19) United States
(12) Patent Application Publication
(10) Pub. No.: US 2004/0152730 A1
(43) Pub. Date: Aug. 5, 2004

(54) Novel compounds

(76) Inventors: Carlo Farina, Baranzate di Bollate (IT); Giuseppe Arnaldo Maria Giardina, Baranzate di Bollate (IT); Mario Grugni, Baranzate di Bollate (IT); Lorenzo Perugini, Verona (IT)

Correspondence Address: SMITHKLINE BEECHAM CORPORATION, CORPORATE INTELLECTUAL PROPERTY-US, UW2220, P. O. BOX 1539, KING OF PRUSSIA, PA, 19406-0939, US

(21) Appl. No.: 10/474,556

(22) PCT Filed: Apr. 11, 2002
(86) PCT No.: PCT/EP02/04069

(30) Foreign Application Priority Data
Apr. 11, 2001 (GB) 0109122.2

(51) Int. Cl. A61K 31/4709; C 07D 4 1/14

(52) U.S. Cl. 514/314; 546/169

(57) ABSTRACT

A compound of formula (I) as detailed in the specification or a pharmaceutically acceptable salt or solvate thereof, a process for preparing such compounds, a pharmaceutical composition comprising such compounds and the use of such compounds in medicine.

The present invention relates to novel compounds, in particular to novel quinoline derivatives, to processes for the preparation of such compounds, to pharmaceutical compositions containing such compounds and to the use of such compounds in medicine.

The mammalian peptide Neurokinin B (NKB) belongs to the Tachykini (TK) peptide family which also include Substance P (SP) and Neurokinin A (NKA). Pharmacological and molecular biological evidence has shown the existence of three subtypes of TK receptor (NK₁, NK₂ and NK₃) and NKB binds preferentially to the NK₃ receptor although it also recognises the other two receptors with lower affinity (Maggi et al, 1993, J. Auton. Pharmacol., 13, 23-93).

Selective peptidic NK₃ receptor antagonists are known (Drapeau, 1990 Regul. Pept., 31, 125-135), and findings with peptidic NK₃ receptor agonists suggest that NKB, by activating the NK₃ receptor, has a key role in the modulation of neural input in airways, skin, spinal cord and nigro-striatal pathways (Myers and Undem, 1993, J.Physiol., 470, 665-679; Counture et al., 1993, Regul. Peptides, 46, 426429; Mccarson and Krause, 1994, J. Neurosci., 14 (2), 712-720; Arenas et al. 1991, J. Neurosci., 11, 2332-8). However, the peptide-like nature of the known antagonists makes them likely to be too labile from a metabolic point of view to serve as practical therapeutic agents.

International Patent Application, Publication Number WO 00/58307 describes a series of aryl fused 2,4-disubstituted pyridines, such as naphthyridine derivatives, which are stated to exhibit biological activity as NK₃ receptor antagonists.

The compounds of the present invention are quinoline derivatives. Other quinoline derivatives have been described previously as selective NK₃ antagonists. For example, International Patent Application, Publication Numbers, WO 95/32948 and WO 96/02509 describe a series of selective and potent NK₃ receptor antagonists.

International Patent Application, Publication Number WO 00/64877 describes a series of 2-aminoquinolinecarboxamides as neurokinin receptor ligands.

International Patent Application, Publication Number, WO 00/58303 describes a series of 4-substituted quinoline derivatives which are stated to be NK₃ and/or GABA(A) receptor ligands. Such compounds are characterised by the presence of a nitrogen-containing heterocyclic moiety at the C(4) position of the quinoline ring.

International Patent Application, Publication Numbers, WO 97/21680, WO 98/52942, WO 00/31037 and WO 00/31038 describe compounds which have biological activity as combined NK₃ and NK₂ receptor antagonists.

Copending International Patent Application Numbers, PCT/EP01/13833, PCT/EP01/14140 and PCT/EP01/13832 also describe compounds that have biological activity as combined NK₃ and NK₂ receptor antagonists.

We have now discovered a further novel class of non-peptide NK₃ antagonists which are far more stable from a metabolic point of view than the known peptidic NK₃ receptor antagonists and are of potential therapeutic utility. These compounds also have NK₂ antagonist activity and are therefore considered to be of potential use in the prevention and treatment of a wide variety of clinical conditions, which are characterised by overstimulation of the Tachykinin receptors, in particular NK₃ and NK₂.

These conditions include respiratory diseases, such as chronic obstructive pulmonary disease (COPD), asthma, airway hyper-reactivity, cough; inflammatory diseases such as inflammatory bowel disease, psoriasis, fibrositis, osteoarthritis, rheumatoid arthritis and inflammatory pain; neurogenic inflammation or peripheral neuropathy, allergies such as eczema and rhinitis; ophthalmic diseases such as ocular inflammation, conjunctivitis, vernal conjuctivitis and the like; cutaneous diseases, skin disorders and itch, such as cutaneous wheal and flare, contact dermatitis, atopic dermatitis, urticaria and other eczematoid dermatitis; adverse immunological reactions such as rejection of transplanted tissues and disorders related to immune enhancement or suppression such as systhemic lupus erythematosis; gastrointestinal (GI) disorders and diseases of the GI tract such as disorders associated with the neuronal control of viscera such as ulcerative colitis, Crohn's disease, irritable bowel syndrome (IOBS), gastro-exophageous reflex disease (GERD); urinary incontinence and disorders of the bladder function; renal disorders; increased blood pressure, proteinuria, coagulopathy and peripheral and cerebral oedema following pre-eclampsia in pregnancies (hereinafter referred to as the ‘Primary Conditions’).

Certain of these compounds also show CNS activity and hence are considered to be of particular use in the treatment of disorders of the central nervous system such as anxiety, depression, psychosis and schizophrenia; neurodegenerative disorders such as AIDS related dementia, senile dementia of the Alzheimer type, Alzheimer's disease, Down's syndrome, Huntingdon's disease, Parkinson's disease, movement disorders and convulsive disorders (for example epilepsy); demyelinating diseases such as multiple sclerosis and amyotrophic lateral sclerosis and other neuropathological disorders such as diabetic neuropathy, AIDS related neuropathy, chemotherapy-induced neuropathy and neuralgia; addiction disorders such as alcoholism; stress related somatic disorders; reflex sympathetic dystrophy such as shoulder/hand syndrome; dysthymic disorders; eating disorders (such as food intake disease); fibrosing and collagen diseases such as scleroderma and eosinophilic fascioliasis; disorders of the blood flow caused by vasodilatation and vasospastic diseases such as angina, migraine and Reynaud's disease and pain or nociception, for example, that is attributable to or associated with any of the foregoing conditions especially the transmission of pain in migraine, (hereinafter referred to as the ‘Secondary Conditions’).

The compounds of formula (I) are also considered to be useful as diagnostic tools for assessing the degree to which neurokinin-3 and neurokinin-2 receptor activity (normal, overactivity or underactivity) is implicated in a patient's symptoms.

Certain compounds of the present invention have also been found to exhibit surprisingly advantageous pharmacochemnical properties.

According to the present invention, there is provided a compound of formula (I) below or a pharmaceutically acceptable salt or solvate thereof:

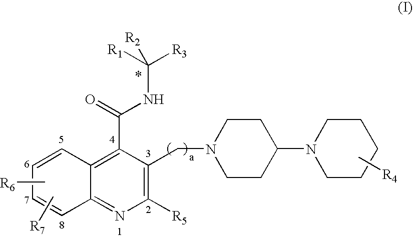

wherein:

R₁ is H or alkyl, R₂ is R₈R₉, and R₃ is H, alkyl or cycloalkyl, optionally substituted by one or more fluorines; or R₂ is R₈R₉ and R₁ and R₃ together with the carbon atom to which they are attached form a cycloalkyl, aryl or heterocyclic ring having 4-7 ring members, which ring R₁/R₃ is unsubstituted or is substituted one or more times by one or more of oxo, hydroxy, halogen, nitro, cyano, carboxy, and amino; or R₃ is H and and R₁ and R₂ together with the carbon atom to which they are attached form a 4-7 membered cycloalkyl, aryl or heterocyclic ring, which cycloalkyl, aryl or heterocyclic ring R₁/R₂ is unsubstituted or is substituted one or more times by one or more substituents selected from alkyl, halo, hydroxy, amino, cyano, nitro, carboxy and oxo, and/or is fused with a cycloalkyl, aryl or 4-7-membered heterocyclic ring;

R₈ represents a single bond or alkyl, optionally substituted by one or more fluorines; R₉ represents an aryl ring or a cycloalkyl or heterocyclic ring having 3-10 ring members, which aryl, cycloalkyl or heterocyclic ring R₉ is unsubstituted or is substituted by R₁₀, which aryl, cycloalkyl or heterocyclic ring R₉ is optionally fused with an aryl, cycloalkyl or 4-7-membered heterocyclic ring;

R₁₀ represents one or more ring substituents independently selected from oxo, hydroxy, halogen, nitro, cyano, carboxy, amino; and/or branched or linear alkyl, alkenyl, alkoxy, or aryl, or a hydroxylated derivative thereof; and/or a branched or linear C₁₋₆ alkyl chain, optionally including one or more of amino, amido, ether, ester, carboxy, sulfonyl, alkenyl, alkynyl, cycloalkyl or aryl functionality and optionally substituted one or more times by one or more of oxo, hydroxy, halogen, nitro, cyano, carboxy, and amino; and/or R₁₀ represents a bridging moiety which is arranged to bridge two ring members in said aryl, cycloalkyl or heterocyclic ring, which bridging moiety comprises mono- or di-oxyalkylene or alkyl;

R₄ represents H or one or more fluorine substituents;

R₅ is branched or linear alkyl, cycloalkyl, cycloalkylalkyl, aryl, or a single or fused ring aromatic heterocyclic group;

R₆ represents H or up to three substituents independently selected from the list consisting of: alkyl, alkenyl, aryl, alkoxy or a hydroxylated derivative thereof, hydroxy, halogen, nitro, cyano, carboxy, alkylcarboxy, alkylcarboxyalkyl, trifluoromethyl, amino or mono- or di- alkylamino; or R₆ represents a bridging moiety which is arranged to bridge two adjacent ring atoms, which bridging moiety comprises alkyl or dioxyalkylene;

R₇ is H, alkoxy or halo;

a is 1-6; and

R₂ or R₅ may optionally be substituted one or more times by halo, hydroxy, amino, cyano, nitro, carboxy or oxo;

not being a compound in which R₄ is H, R₅ is unsubstituted phenyl, R₇ is H, a is 1, and R₁, R₂, R₃ and R₆ are selected from the following:

| 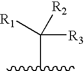 | R₆ |
|---|---|
| 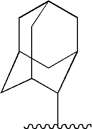 | H |
| 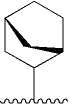 | H |
| 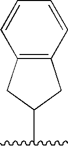 | H |
| 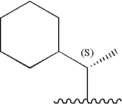 | H |
| 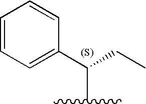 | H |
| 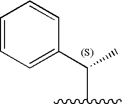 | H |
| 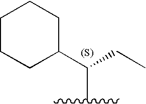 | H |
| 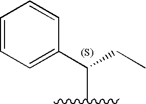 | 7-OMe, 8-Br |
| 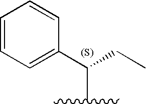 | 7-OMe |
| 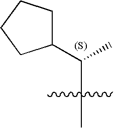 | H |
| 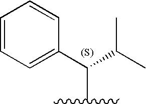 | H |
| 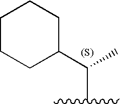 | 7-OMe |
| 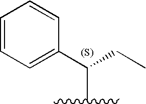 | 7-OH, 8-Cl |
| 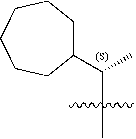 | H |
| 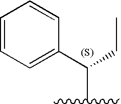 | 7-OH |
| 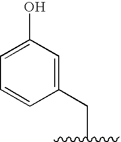 | H |
| 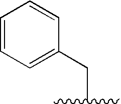 | H |
| 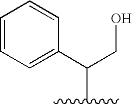 | H |
| 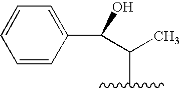 | H |
| 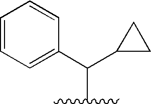 | H |
| 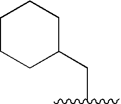 | H |
| 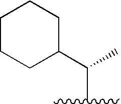 | 6-OH, 7-OH |
| 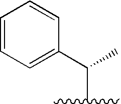 | 6-OH, 7-OH |
| 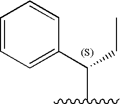 | 6-OEtOH, 7-OEtOH |
| 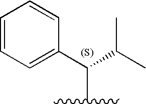 | 6-OH, 7-OH |
| 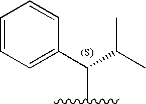 | 6-OMe, 7-OMe |
| 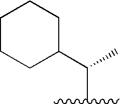 | 6-Cl, 7-Cl, 7-F, 8-F |
| 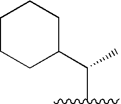 | 6-CF₃, 7-CF₃ |

with the further proviso that said compound of formula (I) is not a compound selected from the following:

3-[1,4′]Bipiperidinyl-1′-ylmethyl-2-thiophen-2-yl-quinoline-4-carboxylic acid ((S)-1-cyclohexyl-ethyl)-amide;

3-[1,4′]Bipiperidinyl-1′-ylmethyl-2-(4-fluoro-phenyl)-quinoline-4-carboxylic acid ((S)-1-cyclohexyl-ethyl)-amide;

3-[1,4′]Bipiperidinyl-1′-ylmethyl-2-(4-trifluoromethyl-phenyl)-quinoline-4-carboxylic acid ((S)-1-cyclohexyl-ethyl)-amide; and

3-[1,4′]Bipiperidinyl-1′-ylmethyl-2-(2-fluoro-phenyl)-quinoline-4-carboxylic acid ((S)-1-cyclohexyl-ethyl)-amide.

In one aspect of the present invention, R₂ is R₈R₉ and R₈ represents a single bond or methyl.

In some such embodiments, R₉ may represent phenyl, or cyclohexyl, or a saturated or unsaturated heterocyclic ring having 5 or 6 ring members and including one or more heteroatoms selected from N, O and S.

Optionally, R₉ may be substituted by R₁₀, and R₁₀ may include 1-3 ring substituents selected from bromo, chloro, fluoro, methyl, ethyl, methoxy, ethoxy, phenyl and cyclohexyl, each of which substituents may optionally be substituted one or more times by halo such as fluoro. In particular, R₁₀ may include one ring substituent which is trifluoromethyl. Alternatively, R₁₀ may include one ring substituent which is branched or linear alkoxy, alkylcarboxy, alkylamino, alkylsulfonyl, alkylether, or alkyloxyamido, which ring substituent is linked to R₉ by a single bond or by C₁₋₃ alkyl. As yet a further alternative, R₁₀ may include one ring substituent which is a bridging moiety comprising ethyl or dioxyethylene.

In other such embodiments, R₉ may represent an aryl, cycloalkyl or 3-10-membered heterocyclic ring which is fused to a phenyl or cyclohexyl ring.

Where R₂ is R₈R₉, R₁ and R₃ together with the carbon atom to which they are attached may form a 5- or 6-membered heterocyclic ring R₁/R₃ comprising one or more heteroatoms selected from N, O and S. Said heterocyclic ring R₁/R₃ may comprise five ring members including two O heteroatoms.

Alternatively, where R₂ is R₈R₉, R₃ may represent methyl, ethyl, iso-propyl or phenyl. In such embodiments, R₁ may advantageously be H or methyl.

In another aspect of the present invention, R₃ may be H and and R₁ and R₂ together with the carbon atom to which they are attached may form a 5-7 membered heterocyclic ring R₁/R₂ comprising one heteroatom selected from N, O and S. Suitably, said heterocyclic ring R₁/R₂ may be substituted one or more times by one or more substituents selected from oxo, methyl and ethyl.

Advantageously, R₅ may be unsubstituted phenyl.

Suitably, R₆ may represent hydrogen, chloro or bromo. Alternatively, R₆ may represent one ring substituent, which is hydroxy, methoxy, ethoxy or a hydroxy-terminated derivative of methoxy or ethoxy, or carboxy or methylcarboxy or ethylcarboxy. Said one ring substituent may suitably be located at the 6 or 7 position around said ring. As yet a further alternative, R₆ may represent a bridging moiety which is arranged to bridge two adjacent ring atoms, which bridging moiety comprises dioxymethylene or dioxyethylene. Said bridging moiety may be arranged to bridge the 6 and 7 positions around said ring.

Preferably, R₇ may represent hydrogen.

Advantageously, a may be 1, 2 or 3. Suitably, a may be 1.

Suitably, R4 is H.

In especially preferred embodiments, a is 1, R₁ is H, R₃ is H, R₄ is H, R₅ is unsubstituted phenyl, R₆ is H, R₇ is H, and R₂ is selected from the following:

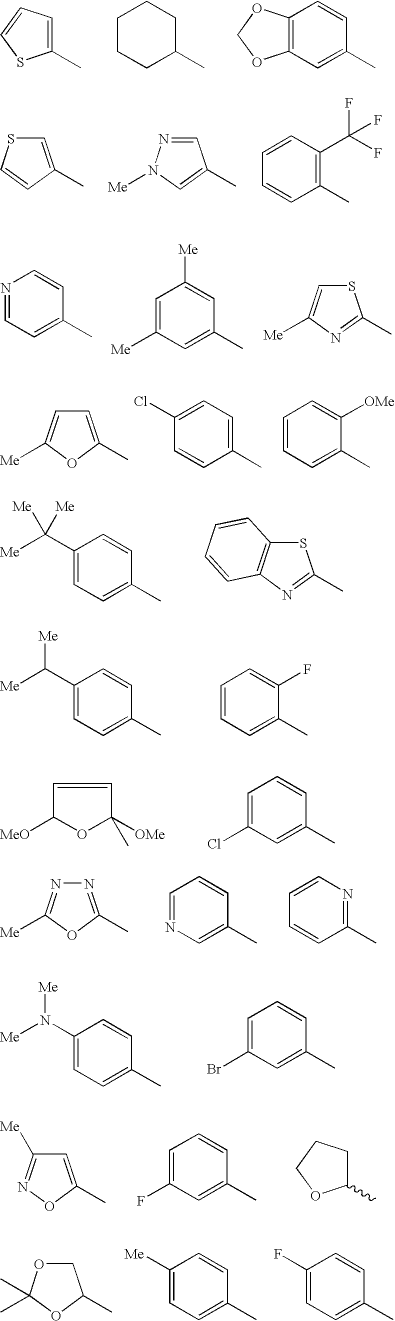

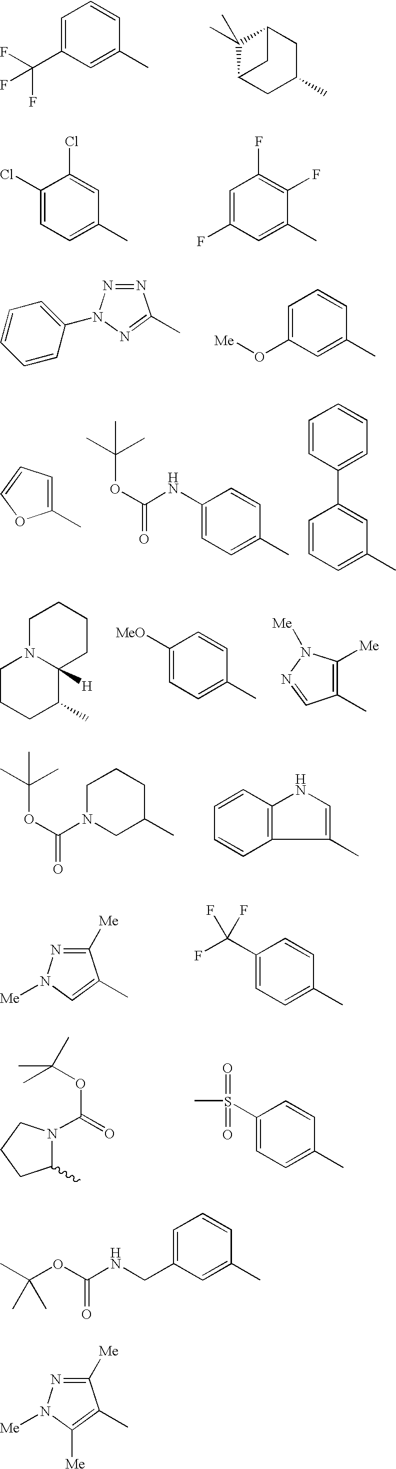

In other especially preferred embodiments, a is 1, R₄ is H, R₅ is unsubstituted phenyl, R₆ is H, R₇ is H, and R₁, R₂ and R₃ are selected from the following:

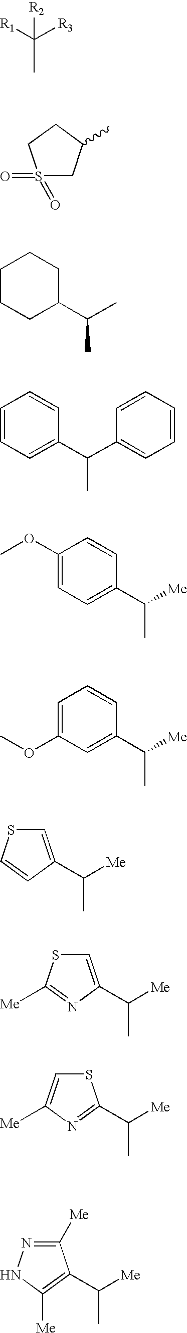

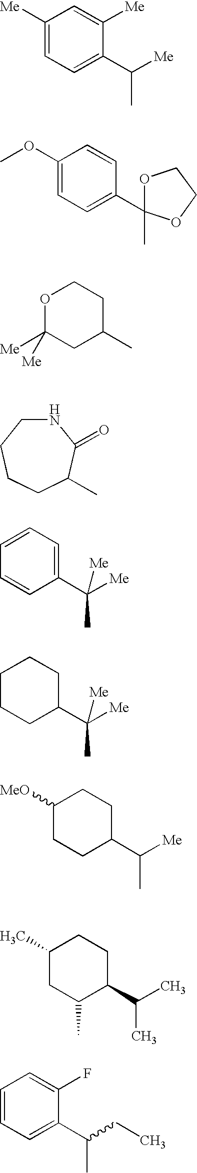

In yet other especially preferred embodiments, the compound of the present invention is selected from the following:

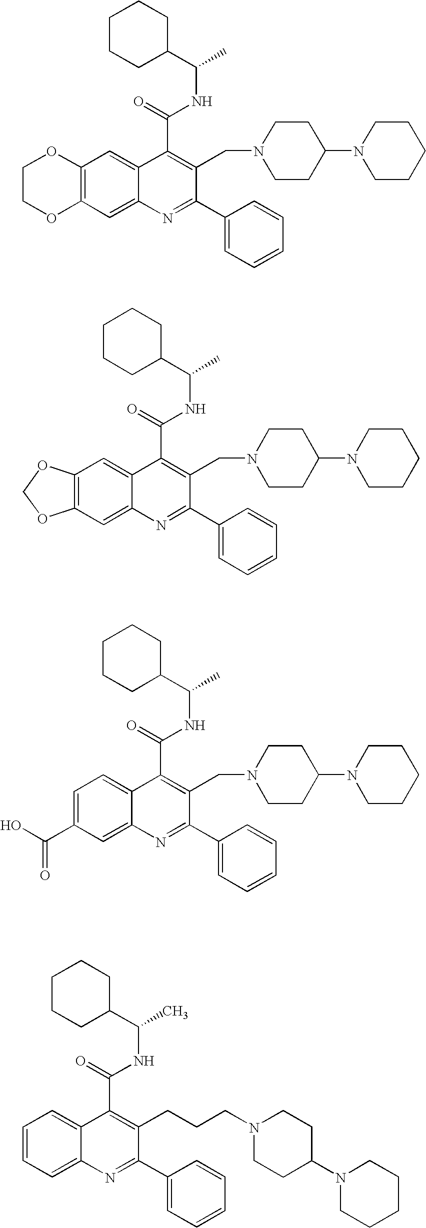

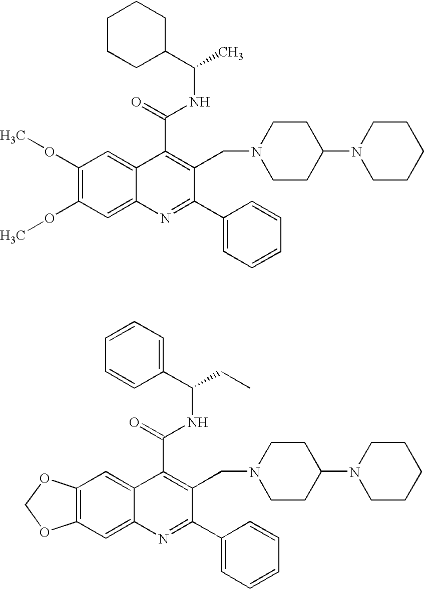

The compounds of formula (I) may have at least one asymmetric centre—for example the carbon atom labelled with an asterisk (*) in the compound of formula (I)—and therefore may exist in more than one steroisomeric form. The invention extends to all such stereoisomeric forms and to mixtures thereof, including racemates. In particular, the invention includes compounds wherein the asterisked carbon atom in formula (I) has the stereochemistry shown in formula (Ia):

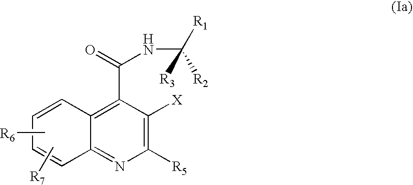

wherein R₁, R₂, R₃, R₅, R₆, and R₇ are as defined in relation to formula (I), and X represents the moiety

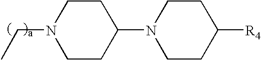

The compounds of formula (I) or their salts or solvates are preferably in pharmaceutically acceptable or substantially pure form. By pharmaceutically acceptable form is meant, inter alia, having a pharmaceutically acceptable level of purity excluding normal pharmaceutical additives such as diluents and carriers, and including no material considered toxic at normal dosage levels.

A substantially pure form will generally contain at least 50% (excluding normal pharmaceutical additives), preferably 75%, more preferably 90% and still more preferably 95% of the compound of formula (I) or its salt or solvate.

One preferred pharmaceutically acceptable form is the crystalline form, including such form in pharmaceutical composition. In the case of salts and solvates the additional ionic and solvent moieties must also be non-toxic.

Suitable salts are pharmaceutically acceptable salts.

Suitable pharmaceutically acceptable salts include the acid addition salts with the conventional pharmaceutical acids, for example maleic, hydrochloric, hydrobromic, phosphoric, acetic, fumaric, salicylic, citric, lactic, mandelic, tartaric, succinic, benzoic, ascorbic and methanesulphonic.

Suitable pharmaceutically acceptable salts include salts of acidic moieties of the compounds of formula (I) when they are present, for example salts of carboxy groups or phenolic hydroxy groups.

Suitable salts of acidic moieties include metal salts, such as for example aluminium, alkali metal salts such as lithium, sodium or potassiumn, alkaline earth metal salts such as calcium or magnesium and ammonium or substituted ammonium salts, for example those with lower alkylamines such as triethylamine, hydroxy alkylamines such as 2-hydroxyethylamine, bis-(2-hydroxyethyl)-amine or tri-(2-hydroxyethyl)-amine, cycloalkylamines such as bicyclohexylamine, or with procaine, dibenzylpiperidine, N-benzyl-β-phenethylamine, dehydroabietylamine, N,N′-bisdehydroabietylamine, glucamine, N-methylglucamine or bases of the pyridine type such as pyridine, collidine, quinine or quinoline.

Suitable solvates are pharmaceutically acceptable solvates.

Suitable pharmaceutically acceptable solvates include hydrates.

The term ‘alkyl’ (unless specified to the contrary) when used alone or when forming part of other groups (such as the ‘alkoxy’ group) includes straight- or branched-chain alkyl groups containing 1 to 12, preferably 1-6 carbon atoms, examples include methyl, ethyl, n-propyl, isopropyl, n-butyl, isobutyl or tert-butyl group.

The term ‘cycloalkyl’ (unless specified to the contrary) when used alone or when forming part of other groups (such as the ‘cycloalkylalkyl’ group) includes cyclic saturated or unsaturated carbon rings including 3-12, preferably 3-8 carbon ring members. Examples include cyclopropyl, cyclobutyl, cyclohexyl, cyclooctyl.

The term ‘alkenyl’ (unless specified to the contrary) when used alone or when forming part of other groups includes straight- or branched- unsaturated carbon chains including at least one double C═C bond and containing 2-12, preferably 2-6 carbon atoms.

The term ‘carbocyclic’ refers to cycloalkyl and aryl rings.

The term ‘aryl’ includes phenyl and naphthyl, preferably phenyl which unless specified to the contrary optionally comprise up to five, preferably up to three substituents selected from halo, alkyl, phenyl, alkoxy, haloalkyl, hydroxyalkyl, hydroxy, amino, nitro, cyano, carboxy, alkoxycarbonyl, alkoxycarbonylalkyl, alkylcarbonyloxy, or alkylcarbonyl groups.

The term ‘aromatic heterocyclic group’ includes groups comprising aromatic heterocyclic rings containing from 5 to 12 ring atoms, suitably 5 or 6, and comprising up to four hetero-atoms in the or each ring selected from S, O or N.

Composite terms such as ‘alkylcarboxy’, ‘cycloalkylalkyl’ and so forth refer to components of a compound which include two interlinked groups, with the group named latterly in the term being the linking group, so that ‘alkylcarboxy’ means (alkyl)—COO— whilst ‘cycloalkylalkyl’ means (cycloalkyl)-(alkyl)-.

Unless specified to the contrary, suitable substituents for any heterocyclic group includes up to 4 substituents selected from the group consisting of: alkyl, alkoxy, aryl and halo or any two substituents on adjacent carbon atoms, together with the carbon atoms to which they are attached, may form an aryl group, preferably a benzene ring, and wherein the carbon atoms of the aryl group represented by the said two substituents may themselves be substituted or unsubstituted.

It will be understood that, unless otherwise specified, groups and substituents forming part of a compound in accordance with the invention are unsubstituted.

When used herein the term “halogen” or “halo” refers to fluorine, chlorine, bromine and iodine, preferably fluorine, chlorine or bromine.

When used herein the term “acyl” includes residues of acids, in particular a residue of a carboxylic acid such as an alkyl- or aryl- carbonyl group.

The invention also provides a process for the preparation of a compound of formula (I), or a salt thereof and/or a solvate thereof, which process comprises reacting a compound of formula (II) or an active derivative thereof:

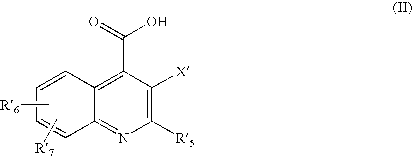

wherein R′₆, R′₇, R′₅ and X′ are R₆, R₇, R₅ and X respectively as hereinbefore defined in relation to formula (I) or (Ia), or a group convertible to R₆, R₇, R₅ and X respectively; with a compound of formula (III):

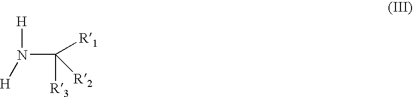

wherein R₁′, R₂′; and R₃′ are R₁, R₂, and R₃ as defined for formula (I) or a group or atom convertible to R₁, R₂, and R₃ respectively; to form a compound of formula (Ib):

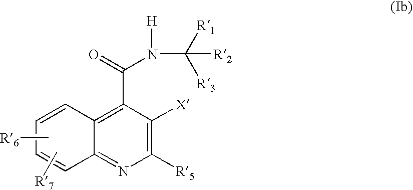

wherein R′₁, R′₂, R′₃, X′, R′₅, R′₆ and R′₇ are as defined above, and thereafter carrying out one or more of the following optional steps:

(i) converting any one of R′₁, R′₂, R′₃, X′, R′₅, R′₆ and R′₇ to R₁, R₂, R₃, X, R₅, R₆ and R₇ respectively as required, to obtain a compound of formula (I);

(ii) converting a compound of formula (I) into another compound of formula (I); and

(iii) preparing a salt of the compound of formula (I) and/or a solvate thereof.

Suitable groups convertible into other groups include protected forms of said groups.

Suitably R′₁, R′₂, R′₃, X′, R′₅, R′₆ and R′₇ each represents R₁, R₂, R₃, X, R₅, R₆ and R₇ respectively or a protected form thereof.

It is favoured if the compound of formula (II) is present as an active derivative.

A suitable active derivative of a compound of formula (II) is a transient activated form of the compound of formula (II) or a derivative wherein the carboxy group of the compound of formula (II) has been replaced by a different group or atom, for example by an acyl halide, preferably a chloride, or an acylazide or a carboxylic acid anhydride.

Other suitable active derivatives include: a mixed anhydride formed between the carboxyl moiety of the compound of formula (II) and an alkyl chloroformate; an activated ester, such as a cyanomethyl ester, thiophenyl ester, p-nitrophenyl ester, p-nitrothiophenyl ester, 2,4,6-trichlorophenyl ester, pentachlorophenyl ester, pentafluorophenyl ester, N-hydroxy-phtalimido ester, N-hydroxypiperidine ester, N-hydroxysuccinimide ester, N-hydroxy benzotriazole ester; alternatively, the carboxy group of the compound of formula (II) may be activated using a carbodiimide or N,N′-carbonyldiimidazole.

The reaction between the compound of formula (II) or the active derivative thereof and the compound of formula (III) is carried out under the appropriate conventional conditions for the particular compounds chosen. Generally, when the compound of formula (II) is present as an active derivative the reaction is carried out using the same solvent and conditions as used to prepare the active derivative, preferably the active derivative is prepared in situ prior to forming the compound of formula (Ib) and thereafter the compound of formula (I) or a salt thereof and/or a solvate thereof is prepared.

For example, the reaction between an active derivative of the compound of formula (II) and the compound of formula (III) may be carried out:

(a) by first preparing an acid chloride and then coupling said chloride with the compound of formula (III) in the presence of an inorganic or organic base in a suitable aprotic solvent such as dimethylformamide (DMF) at a temperature in a range from −70 to 50° C. (preferably in a range from −10 to 20° C.); or

(b) by treating the compound of formula (II) with a compound of formula (III) in the presence of a suitable condensing agent, such as for example N,N′-carbonyl diimidazole (CDI) or a carbodiimide such as dicyclohexylcarbodiimide (DCC) or N-dimethylaminopropyl-N′-ethylcarbodiimide, preferably in the presence of N-hydroxybenzotriazole (HOBT) to maximise yields and avoid racemization processes (see Synthesis, 453, 1972), or O-benzotriazol-1-yl-N,N,N′,N′-tetramethyluroniumhexafluorophosphate (HBTU), in an aprotic solvent, such as a mixture of acetonitrile (MeCN) and tetrahydrofiran (THF), for example a mixture in a volume ratio of from 1:9 to 7:3 (MeCN:THF), at any temperature providing a suitable rate of formation of the required product, such as a temperature in the range of from −70 to 50° C., preferably in a range of from −10 to 25° C., for example at 0° C.

A preferred reaction is set out in Scheme 1a shown below:

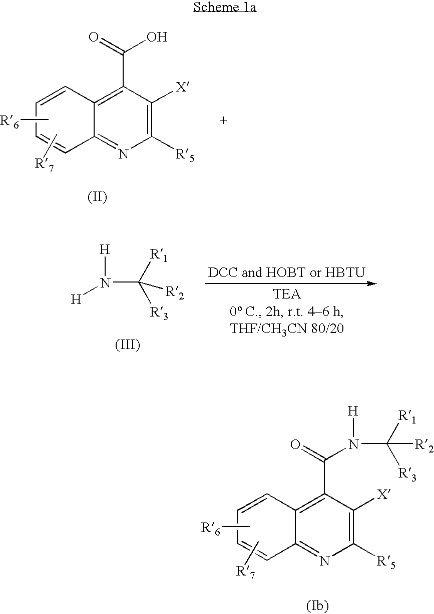

wherein R′₁, R′₂, R′₃, X′, R′₅, R′₆ and R′₇ are as defined above.

Another preferred reaction is set out in Scheme 1b shown below:

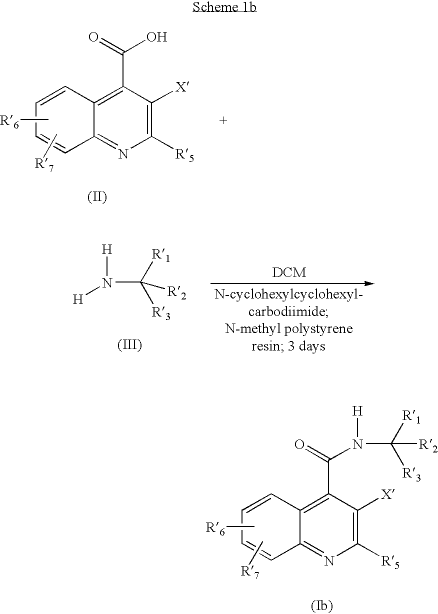

wherein R′₁, R′₂, R′₃, X′, R′₅, R′₆ and R′₇ are as defined above.

It will be appreciated that a compound of formula (Ib) may be converted to a compound of formula (I), or one compound of formula (I) may be converted to another compound of formula (I) by interconversion of suitable substituents. Thus, certain compounds of formula (I) and (Ib) are useful intermediates in forming other compounds of the present invention.

Accordingly, in a further aspect the invention provides a process for preparing a compound of formula (I), or a salt thereof and/or a solvate thereof, which process comprises converting a compound of the above defined formula (Ib) wherein at least one of R₁, R′₂, R′₃, X′, R′₅, R′₆ and R′₇ is not R₁, R₂, R₃, X, R₅, R₆ or R₇ respectively, thereby to provide a compound of formula (I); and thereafter, as required, carrying out one or more of the following optional steps:

(i) converting a compound of formula (I) into another compound of formula (I); and

(ii) preparing a salt of the compound of formula (I) and/or a solvate thereof.

Suitably, in the compound of formula (Ib) the variables R′₁, R′₂, R′₃, X′, R′₅, R′₆ and R′₇ are R₁, R₂, R₃, X, R₅, R₆ and R₇ respectively or they are protected forms thereof.

The above mentioned conversions, protections and deprotections are carried out using the appropriate conventional reagents and conditions and are further discussed below.

In some embodiments, a compound of formula (II) or the corresponding alkyl (such as methyl or ethyl) ester, may be prepared by reacting a compound of formula (IV) or the corresponding alkyl (such as methyl or ethyl) ester:

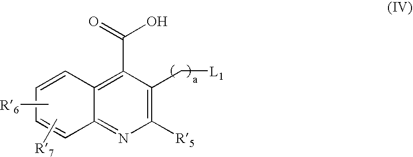

wherein R′₆, R′₇, R′₅ and a are as defined above and L₁ represents a halogen atom such as a bromine atom, with a compound of formula (V):

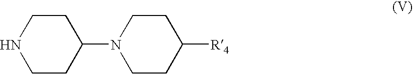

wherein R′₄ is R₄ as defined in relation to formula (I) or a protected form thereof

Suitably, R′₄ is R₄.

Suitably, reaction between the compounds of formulae (IV) or the corresponding allyl (such as methyl or ethyl) ester and (V) is carried out under conventional amination conditions, for example when L₁ is a bromine atom then the reaction is conveniently carried out in an aprotic solvent, such as tetrahydrofuran or dimethylformamide at any temperature providing a suitable rate of formation of the required product, usually at ambient temperature; preferably the reaction is carried out in the presence of triethylamine (TEA) or K₂CO₃.

Where a is 1, a compound of formula (IV) or the corresponding alkyl (such as methyl or ethyl) ester can be prepared by appropriate halogenation of a compound of formula (VI) or the corresponding alkyl (such as methyl or ethyl) ester:

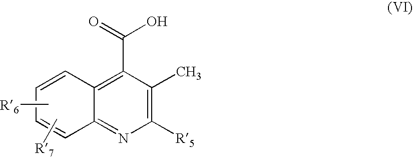

wherein R′₆, R′₇ and R′₅ are as defined above in relation to formula (II).

Suitable halogenation reagents are conventional reagents depending upon the nature of the halogen atom required, for example when L₁ is bromine a preferred halogenation reagent is N-bromosuccinimide (NBS).

The halogenation of the compound of formula (VI) or the corresponding alkyl (such as methyl or ethyl) ester is preferably carried out under conventional conditions, for example bromination is carried out by treatment with NBS in an inert solvent, such as carbon tetrachloride CCl₄, or 1,2-dichloroethane or CH₃CN, at any temperature providing a suitable rate of formation of the required product, suitably at an elevated temperature such as a temperature in the range of 60° C. to 100° C., for example 80° C.; preferably the reaction is carried out in the presence of a catalytic amount of benzoyl peroxide.

A compound of formula (VI) may conveniently be prepared by reacting a compound of formula (A)

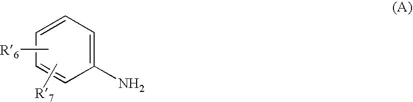

wherein R′₆ and R′₇ are as defined in relation to formula (II), with a compound of formula (B):

R′₅—CHO (B)

wherein R′₅ is as defined in relation to formula (II), in the presence of oxobutyric acid.

The reaction between the compounds of formula (A) and (B) respectively is conveniently carried out using Doebner-Miller reaction conditions (see for example Chem. Ber. 29, 352 (1894); Chem. Revs. 35, 153, (1944); J. Chem. Soc. B, 1969, 805), for example in an alcoholic solvent such as ethanol, at any temperature providing a suitable rate of formation of the required product, but generally at an elevated temperature, such as the reflux temperature of the solvent.

The compounds of formula (A) and (B) are known compounds or they are prepared according to methods used to prepare known compounds for example as described in Vogel's Textbook of practical Organic Chemistry.

Alternatively a compound of formula (VI) can be prepared by reacting a compound of formula (VII)

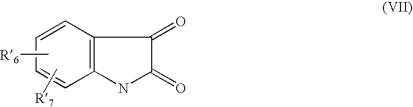

wherein R′₆ and R′₇ are as defined in relation to formula (II), with a compound of formula (C):

R₅′—CO—CH₂—CH₃ (C)

The reaction between the compounds of formula (VII) and (C) respectively is conveniently carried out using Pfitzinger reaction conditions (see for example J. Prakt. Chem. 33, 100 (1886), J. Prakt. Chem. 38, 582 (1888), J. Chem. Soc. 106 (1948) and Chem. Rev. 35, 152 (1944)), for example in an alkanolic solvent such as ethanol, at any temperature providing a suitable rate of formation of the required product, but generally at an elevated temperature, such as the reflux temperature of the solvent, and preferably in the presence of a base such as potassium hydroxide or potassium tert-butoxide. The compounds of formula (C) are known compounds or they are prepared according to methods used to prepare known compounds for example as described in Vogel's Textbook of Practical Organic Chemistry.

In the case in which the corresponding alkyl (such as methyl or ethyl) ester of compounds (VI), (IV) and (II) are utilised, a hydrolysis to compound (II) is required before conversion to compound (Ib) in Scheme 1a or 1b. Such hydrolysis can be carried out under acidic conditions, such 10-36% hydrochloric acid at a temperature in the range between 30 and 100° C.

In other embodiments, a compound of formula (II) wherein X′ represents

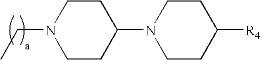

may conveniently be prepared by reacting a compound of formula (VII) with a compound of formula (VIII):

R₅′—CO—CH₂—(CH₂)a-T₅ (VIII)

wherein R′₅ and a are as defined in relation to formula (II), and T₅ is a group X′ as defined in relation to formula (II) or a protected form thereof or a group convertible thereto; and thereafter as required removing any protecting group, for example by dehydrogenation, and/or converting any group T₅ to X as defined in relation to formula (I).

The reaction between the compounds of formula (VII) and (VIII) is conveniently carried out using Pfitzinger reaction conditions (see for example J. Prakt. Chem. 33, 100 (1886), J. Prakt. Chem. 38, 582 (1888), J. Chem. Soc. 106 (1948) and Chem. Rev. 35, 152 (1944)), for example in an alkanolic solvent such as ethanol, at any temperature providing a suitable rate of formation of the required product, but generally at an elevated temperature, such as the reflux temperature of the solvent, and preferably in the presence of a base such as potassium hydroxide or potassium tert-butoxide.

Protected forms of

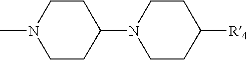

will vary according to the particular nature of the group being protected but will be chosen in accordance with normal chemical practice.

Groups convertible to

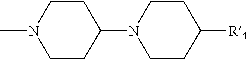

include groups dictated by conventional chemical practice to be required and to be appropriate, depending upon the specific nature of the

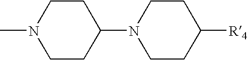

under consideration. Suitable deprotection methods for deprotecting protected forms of

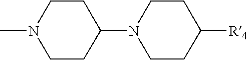

and conversion methods for converting T₅ to

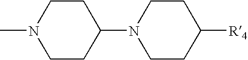

will be those used conventionally in the art depending upon the particular groups under consideration with reference to standard texts such as Greene, T. W. and Wuts, P. G. M. Protective Groups in Organic Synthesis, John Wiley & Sons Inc. New York, 1991 (Second Edt.) or in Kocienski, P. J. Protecting groups. George Thieme Verlag, New York, 1994 and Chemistry of the Amino Group, Patais (Ed.), Interscience, New York 1968; or Advanced Organic Chemistry, March J, John Wiley & Sons, New York, 1992.

A compound of formula (VII is prepared from a compound of formula (IX):

R₅′—CO—CH₂—(CH₂)ₐ—OH (IX)

wherein R′₅ is as defined in relation to formula (II) and a is as defined in relation to formula (VIII), by first halogenating, preferably brominating, or mesylating the compound of formula (IX) and thereafter reacting the halogenation or mesylation product so formed with a compound capable of forming a group T₅ so as to provide the required compound of formula (VII).

When T₅ is a group

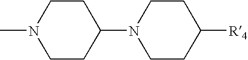

a compound capable of forming a group T₅ is a compound of the above defined formula (V).

The halogenation of the compound of formula (IX) is suitably carried out using a conventional halogenation reagent. Mesylation is conveniently carried out using mesyl chloride in an inert solvent such as methylene dichloride, at a temperature below room temperature, such as 0° C., preferably in the presence of triethylamine. The reaction conditions between the compound of formula (IX) and the compound capable of forming a group T₅ will be those conventional conditions dictated by the specific nature of the reactants, for example when the T₅ required is a group

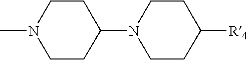

and the required compound capable of forming a group T₅ is a compound of the above defined formula (V), then the reaction between the halogenation or mesylation product of the compound of formula (IX) and the compound of formula (V) is carried out under analogous conditions to those described for the reaction between the compounds of formulae (IV) and (V).

Other compounds capable of forming a group T₅ will depend upon the particular nature of T₅, but will be those appropriate compounds dictated by conventional chemical practice with reference to standard texts such as Chemistry of the Amino Group, Patais (Ed.), Interscience, New York 1968; and Advanced Organic Chemistry, March J, John Wiley & Sons, New York, 1992.

A compound of formula (IX) may be prepared by reacting a compound of formula (X):

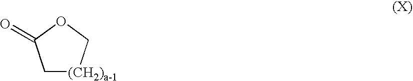

wherein a is as defined in relation to formula (VIII), with a lithium salt of formula (XI):

R′₅Li (XI)

wherein R′₅ is as defined in relation to formula (II).

The reaction between the compounds of formulae (X) and (XI) can be carried out in an aprotic solvent, such as diethyl-ether at any temperature providing a suitable rate of formation of the required product, usually at a low temperature such as in the range of −10° C. to −30° C., for example −20° C.

The compounds of formula (III) are known commercially available compounds or they can be prepared from known compounds by known methods, or methods analogous to those used to prepare known compounds, for example the methods described in Liebigs Ann. der Chemie, (1936), 523, 199.

A chiral compound of formula (III) wherein R₂ is a C₅ or C₇ cycloalkyl group, R₃ is methyl and R₁ is H are described in J. Org. Chem. (1996), 61 (12), 41304135. A chiral compound of formula (III) wherein R₂ is phenyl, R₃ is isopropyl and R₁ is H is a known compound described in for example Tetrahedron Lett. (1994), 35(22), 3745-6.

The compounds of formula (V) are known, commercially available compounds or they can be prepared using methods analogous to those used to prepare known compounds; for example the methods described in the Chemistry of the Amino Group, Patais (Ed.), Interscience, New York 1968; Advanced Organic Chemistry, March J, John Wiley & Sons, New York, 1992 ; J. Heterocyclic Chem. (1990), 27, 1559; Synthesis (1975), 135, Bioorg. Med. Chem. Lett. (1997), 7, 555, or Protective Groups in Organic Synthesis (second edition), Wiley Interscience, (1991) or other methods mentioned herein.

The compounds of formula (VII) are known compounds or they are prepared according to methods used to prepare known compounds for example those disclosed in J. Org. Chem. 21, 171 (1955); J. Org. Chem. 21, 169 (1955).

The compounds of formula (X) and (XI) are known compounds or they are prepared according to methods used to prepare known compounds for example those disclosed by Krow G. R. in Organic Reactions, Vol 43, page 251, John Wiley & Sons Inc.1994 (for the compounds of formula (X)) and Organometallics in Synthesis, Schlosser M.(Ed), John Wiley & Sons Inc.1994 (for the compounds of formula (XI)).

As hereinbefore mentioned, the compounds of formula (I) may exist in more than one stereoisomeric form—and the process of the invention may produce racemates as well as enantiomerically pure forms. Accordingly, a pure enantiomer of a compound of formula (I) is obtained by reacting a compound of the above defined formula (II) with an appropriate enantiomerically pure primary amine of formula (IIIa) or (IIIc):

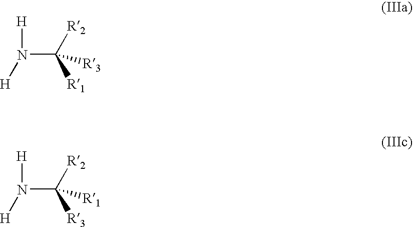

wherein R′₁, R′₂ and R′₃ are as defined above, to obtain a compound of formula (I′a) or (I′c):

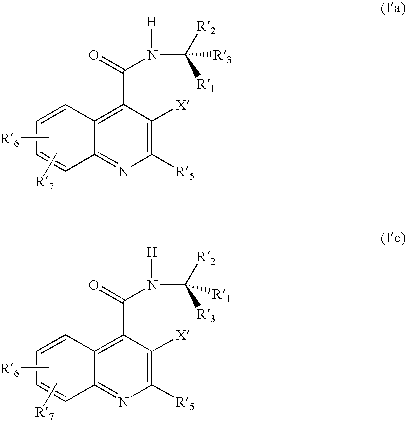

wherein R′₁, R′₂, R′₃, X′, R′₅, R′₆, and R′₇ are as defined above.

Compounds of formula (I′a) or (I′c) may subsequently be converted to compounds of formula (Ia) or (Ic) by the methods of conversion mentioned before:

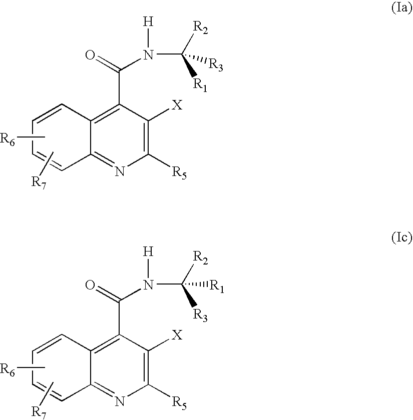

wherein R₁, R₂, R₃, X, R₅, R₆, and R₇ are as defined above.

Suitably, in the above mentioned compounds of formulae (Ia), (Ic), (I′a), (I′c), (IIIa) and (IIIc) R₁ represents hydrogen.

An alternative method for separating optical isomers is to use conventional, fractional separation methods in particular fractional crystallisation methods. Thus, a pure enantiomer of a compound of formula (I) is obtained by fractional crystallisation of a diastereomeric salt formed by reaction of the racemic compound of formula (I) with an optically active strong acid resolving agent, such as camphosulphonic acid, tartaric acid, O,O′-di-p-toluoyltartaric acid or mandelic acid, in an appropriate alcoholic solvent, such as ethanol or methanol, or in a ketonic solvent, such as acetone. The salt formation process should be conducted at a temperature between 20° C. and 80° C., preferably at 50° C.

A suitable conversion of one compound of formula (I) into a further compound of formula (I) involves converting one group X into another group X by for example:

(i) converting a ketal into a ketone, by such as mild acidic hydrolysis, using for example dilute hydrochloric acid;

(ii) reducing a ketone to a hydroxyl group by use of a borohydride reducing agent;

(iii) converting a carboxylic ester group into a carboxyl group using basic hydrolysis; and/or

(iv) reducing a carboxylic ester group to a hydroxymethyl group, by use of a borohydride reducing agent.

As indicated above, where necessary, the conversion of any group R′₁, R′₂, R′₃, X′, R′₅, R′₆, and R′₇ into R₁, R₂, R₃, X, R₅, R₆, and R₇ which as stated above are usually protected forms of R₁, R₂, R₃, X, R₅, R₆, or R₇ may be carried out using appropriate conventional conditions such as the appropriate deprotection procedure.

It will be appreciated that in any of the above mentioned reactions any reactive group in the substrate molecule may be protected and deprotected according to conventional chemical practice, for example as described by Greene, T. W. and Wuts, P. G. M. Protective Groups in Organic Synthesis, John Wiley & Sons Inc. New York, 1991 (Second Edt.) or in Kocienski, P. J. Protecting groups. George Thieme Verlag, New York, 1994.

Suitable protecting groups in any of the above mentioned reactions are those used conventionally in the art. Thus, for example suitable hydroxyl protecting groups include benzyl or trialkylsilyl groups.

The methods of formation and removal of such protecting groups are those conventional methods appropriate to the molecule being protected. Thus for example a benzyloxy group may be prepared by treatment of the appropriate compound with a benzyl halide, such as benzyl bromide, and thereafter, if required, the benzyl group may be conveniently removed using catalytic hydrogenation or a mild ether cleavage reagent such as trimethylsilyl iodide or boron tribromide.

As indicated above, the compounds of formula (I) have useful pharmaceutical properties.

Accordingly the present invention also provides a compound of formula (I), or a pharmaceutically acceptable salt or solvate thereof, for use as an active therapeutic substance.

In particular, the present invention also provides a compound of formula (I), or a pharmaceutically acceptable salt or solvate thereof, for the treatment or prophylaxis of the Primary and Secondary Conditions.

The present invention further provides a pharmaceutical composition comprising a compound of formula (I), or a pharmaceutically acceptable salt or solvate thereof, and a pharmaceutically acceptable carrier.

The present invention also provides the use of a compound of formula (I), or a pharmaceutically acceptable salt or solvate thereof, in the manufacture of a medicament for the treatment of the Primary and Secondary Conditions.

As mentioned above the Primary conditions include respiratory diseases, such as chronic obstructive pulmonary disease (COPD), asthma, airway hyperreactivity, cough; inflammatory diseases such as inflammatory bowel disease, psoriasis, fibrositis, osteoarthritis, rheumatoid arthritis and inflammatory pain; neurogenic inflammation or peripheral neuropathy, allergies such as eczema and rhinitis; ophthalmic diseases such as ocular inflammation, conjunctivitis, vernal conjuctivitis and the like; cutaneous diseases, skin disorders and itch, such as cutaneous wheal and flare, contact dermatitis, atopic dermatitis, urticaria and other eczematoid dermatitis; adverse immunological reactions such as rejection of transplanted tissues and disorders related to immune enhancement or suppression such as systhemic lupus erythematosis; gastrointestinal (GI) disorders and diseases of the GI tract such as disorders associated with the neuronal control of viscera such as ulcerative colitis, Crohn's disease, irritable bowel syndrome (IBS), gastro-exophageous reflex disease (GERD); urinary incontinence and disorders of the bladder function; renal disorders; increased blood pressure, proteinuria, coagulopathy and peripheral and cerebral oedema following pre-eclampsia in pregnancies.

As mentioned above, the Secondary conditions include disorders of the central nervous system such as anxiety, depression, psychosis and schizophrenia; neurodegenerative disorders such as AIDS related dementia, senile dementia of the Alzheimer type, Alzheimer's disease, Down's syndrome, Huntingdon's disease, Parkinson's disease, movement disorders and convulsive disorders (for example epilepsy); demyelinating diseases such as multiple sclerosis and amyotrophic lateral sclerosis and other neuropathological disorders such as diabetic neuropathy, AIDS related neuropathy, chemotherapy-induced neuropathy and neuralgia; addiction disorders such as alcoholism; stress related somatic disorders; reflex sympathetic dystrophy such as shoulder/hand syndrome; dysthymic disorders; eating disorders (such as food intake disease); fibrosing and collagen diseases such as scleroderma and eosinophilic fascioliasis; disorders of the blood flow caused by vasodilatation and vasospastic diseases such as angina, migraine and Reynaud's disease and pain or nociception, for example, that is attributable to or associated with any of the foregoing conditions especially the transmission of pain in migraine.

Such a medicament, and a composition of this invention, may be prepared by admixture of a compound of the invention with an appropriate carrier. It may contain a diluent, binder, filler, disintegrant, flavouring agent, colouring agent, lubricant or preservative in conventional manner.

These conventional excipients may be employed for example as in the preparation of compositions of known agents for treating the conditions.

Preferably, a pharmaceutical composition of the invention is in unit dosage form and in a form adapted for use in the medical or veterinarial fields. For example, such preparations may be in a pack form accompanied by written or printed instructions for use as an agent in the treatment of the conditions.

The suitable dosage range for the compounds of the invention depends on the compound to be employed and on the condition of the patient. It will also depend, inter alia, upon the relation of potency to absorbability and the frequency and route of administration.

The compound or composition of the invention may be formulated for administration by any route, and is preferably in unit dosage form or in a form that a human patient may administer to himself in a single dosage. Advantageously, the composition is suitable for oral, rectal, topical, parenteral, intravenous or intramuscular administration. Preparations may be designed to give slow release of the active ingredient.

Compositions may, for example, be in the form of tablets, capsules, sachets, vials, powders, granules, lozenges, reconstitutable powders, or liquid preparations, for example solutions or suspensions, or suppositories.

The compositions, for example those suitable for oral administration, may contain conventional excipients such as binding agents, for example syrup, acacia, gelatine, sorbitol, tragacanth, or polyvinylpyrrolidone; fillers, for example lactose, sugar, maize-starch, calcium phosphate, sorbitol or glycine; tabletting lubricants, for example magnesium stearate; disintegrants, for example starch, polyvinyl-pyrrolidone, sodium starch glycollate or microcrystalline cellulose; or pharmaceutically acceptable setting agents such as sodium lauryl sulphate.

Solid compositions may be obtained by conventional methods of blending, filling, tabletting or the like. Repeated blending operations may be used to distribute the active agent throughout those compositions employing large quantities of fillers. When the composition is in the form of a tablet, powder, or lozenge, any carrier suitable for formulating solid pharmaceutical compositions may be used, examples being magnesium stearate, starch, glucose, lactose, sucrose, rice flour and chalk. Tablets may be coated according to methods well known in normal pharmaceutical practice, in particular with an enteric coating. The composition may also be in the form of an ingestible capsule, for example of gelatine containing the compound, if desired with a carrier or other excipients.

Compositions for oral administration as liquids may be in the form of, for example, emulsions, syrups, or elixirs, or may be presented as a dry product for reconstitution with water or other suitable vehicle before use. Such liquid compositions may contain conventional additives such as suspending agents, for example sorbitol, syrup, methyl cellulose, gelatine, hydroxyethylcellulose, carboxymethylcellulose, aluminium stearate gel, hydrogenated edible fats; emulsifying agents, for example lecithin, sorbitan monooleate, or acacia; aqueous or non-aqueous vehicles, which include edible oils, for example almond oil, fractionated coconut oil, oily esters, for example esters of glycerine, or propylene glycol, or ethyl alcohol, glycerine, water or normal saline; preservatives, for example methyl or propyl p-hydroxybenzoate or sorbic acid; and if desired conventional flavouring or colouring agents.

The compounds of this invention may also be administered by a non-oral route. In accordance with routine pharmaceutical procedure, the compositions may be formulated, for example for rectal administration as a suppository. They may also be formulated for presentation in an injectable form in an aqueous or non-aqueous solution, suspension or emulsion in a pharmaceutically acceptable liquid, e.g. sterile pyrogen-free water or a parenterally acceptable oil or a mixture of liquids. The liquid may contain bacteriostatic agents, anti-oxidants or other preservatives, buffers or solutes to render the solution isotonic with the blood, thickening agents, suspending agents or other pharmaceutically acceptable additives. Such forms will be presented in unit dose form such as ampoules or disposable injection devices or in multi- dose forms such as a bottle from which the appropriate dose may be withdrawn or a solid form or concentrate which can be used to prepare an injectable formulation.

The compounds of this invention may also be administered by inhalation, via the nasal or oral routes. Such administration can be carried out with a spray formulation comprising a compound of the invention and a suitable carrier, optionally suspended in, for example, a hydrocarbon propellant.

Preferred spray formulations comprise micronised compound particles in combination with a surfactant, solvent or a dispersing agent to prevent the sedimentation of suspended particles. Preferably, the compound particle size is from about 2 to 10 microns.

A further mode of administration of the compounds of the invention comprises transdermal delivery utilising a skin-patch formulation. A preferred formulation comprises a compound of the invention dispersed in a pressure sensitive adhesive which adheres to the skin, thereby permitting the compound to diffuse from the adhesive through the skin for delivery to the patient. For a constant rate of percutaneous absorption, pressure sensitive adhesives known in the art such as natural rubber or silicone can be used.

As mentioned above, the effective dose of compound depends on the particular compound employed, the condition of the patient and on the frequency and route of administration. A unit dose will generally contain from 20 to 1000 mg and preferably will contain from 30 to 500 mg, in particular 50, 100, 150, 200, 250, 300, 350, 400, 450, or 500 mg. The composition may be administered once or more times a day for example 2, 3 or 4 times daily, and the total daily dose for a 70 kg adult will normally be in the range 100 to 3000 mg. Alternatively the unit dose will contain from 2 to 20 mg of active ingredient and be administered in multiples, if desired, to give the preceding daily dose.

No unacceptable toxicological effects are expected with compounds of the invention when administered in accordance with the invention.

The present invention also provides a method for the treatment and/or prophylaxis of the Primary and Secondary Conditions in mammals, particularly humans, which comprises administering to the mammal in need of such treatment and/or prophylaxis an effective, non-toxic pharmaceutically acceptable amount of a compound of formula (I) or a pharmaceutically acceptable salt or solvate thereof.

The activity of the compounds of the present invention, as NK₃ ligands, is determined by their ability to inhibit the binding of the radiolabelled NK₃ ligands, [¹²⁵I] [Me-Phe⁷]-NKB or [³H]-Senktide, to guinea-pig and human NK₃ receptors (Renzetti et al, 1991, Neuropeptide, 18, 104-114; Buell et al, 1992, FEBS, 299(1), 90-95; Chung et al, 1994, Biochem. Biophys. Res. Commun., 198(3), 967-972).

The binding assays utilised allow the determination of the concentration of the individual compound required to reduce by 50% the [¹²⁵I]-[Me-Phe⁷]-NKB and [³I]-Senktide specific binding to NK₃ receptor in equilibrium conditions (IC50).

Binding assays provide for each compound tested a mean IC₅₀ value of 2-5 separate experiments performed in duplicate or triplicate. The most potent compounds of the present invention show IC₅₀ values in the range 0.1-1000 nM. The NK₃-antagonist activity of the compounds of the present invention is determined by their ability to inhibit senktide-induced contraction of the guinea-pig ileum (Maggi et al, 1990, Br. J. Pharmacol., 101, 996-1000) and rabbit isolated iris sphincter muscle (Hall et al., 1991, Eur. J. Pharmacol., 199, 9-14) and human NK₃ receptors-mediated Ca⁺⁺ mobilisation (Mochizuki et al, 1994, J. Biol. Chem., 269, 9651-9658). Guinea-pig and rabbit in-vitro functional assays provide for each compound tested a mean K_{B} value of 3-8 separate experiments, where K_{B} is the concentration of the individual compound required to produce a 2-fold rightward shift in the concentration-response curve of senktide. Human receptor functional assay allows the determination of the concentration of the individual compound required to reduce by 50% (IC₅₀ values) the Ca⁺⁺ mobilisation induced by the agonist NKB. In this assay, the compounds of the present invention behave as antagonists.

The activity of the compounds of the present invention, as NK-2 ligands, is determined by their ability to inhibit the binding of the radiolabelled NK-2 ligands, [125I]-NKA or [³H]-NKA, to human NK-2 receptors (Aharony et al, 1992, Neuropeptide, 23, 121-130).

The binding assays utilised allow the determination of the concentration of the individual compound required to reduce by 50% the [¹²⁵I]-NKA and [³H]-NKA specific binding to NK2 receptor in equilibrium conditions (IC₅₀).

Binding assays provide for each compound tested a mean IC₅₀ value of 2-5 separate experiments performed in duplicate or triplicate. The most potent compounds of the present invention show IC₅₀ values in the range 0.5-1000 nM, such as 1-1000 nM. The NK-2-antagonist activity of the compounds of the present invention is determined by their ability to inhibit human NK-2 receptor-mediated Ca⁺⁺ mobilisation (Mochizuki et al, 1994, J. Biol. Chem., 269, 9651-9658). Human receptor functional assay allows the determination of the concentration of the individual compound required to reduce by 50% (IC₅₀ values) the Ca⁺⁺ mobilisation induced by the agonist NKA. In this assay, the compounds of the present invention behave as antagonists.

The therapeutic potential of the compounds of the present invention in treating the conditions can be assessed using rodent disease models.

As stated above, the compounds of formula (I) are also considered to be useful as diagnostic tools. Accordingly, the invention includes a compound of formula (I) for use as diagnostic tools for assessing the degree to which neurokinin-3 and neurokinin-2 receptor activity (normal, overactivity or underactivity) is implicated in a patient's symptoms. Such use comprises the use of a compound of formula (I) as an antagonist of said activity, for example including but not restricted to Tachykinin agonist-induced inositol phosphate turnover or electrophysiological activation, of a cell sample obtained from a patient. Comparison of such activity in the presence or absence of a compound of formula (I), will disclose the degree of NK-3 and NK-2 receptor involvement in the mediation of agonist effects in that tissue.

The following Descriptions illustrate the preparation of the intermediates, whereas the following Examples illustrate the preparation of the compounds of the invention.

### DESCRIPTIONS AND EXAMPLES

Description 1. 3-Methyl-2-phenyl-quinoline-4-carboxylic acid methyl ester

30 g (114 mmol) of 3-methyl-2-phenyl-quinoline-4-carboxylic acid (CAS [43071-45-0]) were suspended in 250 ml of dry CH₂Cl₂; 20 ml (230 mmol) of oxalyl chloride dissolved in 120 ml of CH₂Cl₂ were added dropwise and the reaction mixture was stirred at room temperature for 30 min. Two drops of N,N-dimethylformamide (DMF) were added and the reaction was stirred for additional 30 min. The solvent was evaporated in vacuo to dryness, the residue was taken up with 100 ml of CH₂Cl₂ and 100 ml of MeOH, dissolved in 400 ml of CH₂Cl₂, were added dropwise. After stirring for 18 h, the solvent was evaporated in vacuo to dryness, the residue was taken up with CH₂Cl₂ and washed with 1% NaHCO₃; the organic layer was dried over Na₂SO₄, filtered and evaporated in vacuo to dryness to yield the title compound as a solid, which may be used without further purification.

C₁₈H₁₅NO₂

MW 277.31

MP=73-75° C.

IR (KBr) 3441, 3051, 2954, 1731, 1582, 1556 cm⁻¹.

Description 2. 3-Bromomethyl-2-phenyl-quinoline-4-carboxylic acid methyl ester

3-Methyl-2-phenyl-quinoline-4-carboxylic acid methyl ester (10 g, 36 mmol), prepared as in Description 1, was dissolved in CH₃CN (500 ml) and N-bromosuccinimide (13 g, 72 mmol) was added. After adding dibenzoylperoxide (1 g, 4.1 mmol), the reaction was refluxed for 24 h; then additional N-bromosuccinimide (4 g, 22.5 mmol) and dibenzoylperoxide (0.5 g, 2.0 mmol) were added and the reaction was refluxed for additional 4 h. The solvent was evaporated in vacuo to dryness to yield the tile compound, which may be used without flrter purification.

C₁₈H₁₄BrNO₂

MW=356.23

Description 3. 3-[1,4′]Bipiperidinyl-1′-ylmethyl-2-phenyl-quinoline-4-carboxylic acid methyl ester

10.5 g (56.14 mmol) of 4-piperidinopiperidine and 7.8 g of K₂CO₃ were suspended in 350 ml CH₃CN. A solution of 20 g (56.14 mmol) 3-bromomethyl-2-phenyl-quinoline-4-carboxylic acid methyl ester (compound of Description 2) in 100 ml of CH₃CN/CH₂Cl₂ (10:1 mixture) was added dropwise at room temperature. The reaction was stirred overnight After all the bromide had been consumed, the suspension was filtered and the obtained solution evaporated. The crude solid was taken up with i-Pr₂O and the insoluble portion was removed by filtration. The liquid phase was then evaporated to dryness and the resulting oil was purified by flash chromatography (eluent AcOEt:MeOH:NH₃ 95:5:0.5) to yield the title compound.

C₂₈H₃₃N₃O₂

MW=443.59

MP=118-120° C.

Description 4. 3-[1,4′]Bipiperidinyl-1′-ylmethyl-2-phenyl-quinoline-4-carboxylic acid

Aminoacid trihydrochloride (8.45 g, 15.67 mmol), obtained by hydrolysis in strongly acidic conditions of the corresponding 3-[1,4′]bipiperidinyl-1′-ylmethyl-2-phenyl-quinoline-4-carboxylic acid methyl ester, was suspended in CH₃CN (150 ml) and 5.88 ml (0.918 g/ml, 47.04 mmol) of tetramethylguanidine were slowly added to the stirred mixture. By the end of the addition the aminoacid was completely dissolved and after a few minutes a white solid precipitated. The solid was filtered and crystallised from CH₃CN, yiedling a solid whose composition, established by ¹H-NMR, MS and IR analysis, resulted in the free aminoacid containing a ˜20%_{WT} of tetramethylguanidine. In different batches tetramethylguanidine was variably detected, as hydrochloride, ranging from 8%_{WT} to 20%_{WT}. The presence of this salt however was found not to interfere with following the synthetic step.

C₂₇H₃₁N₃O₂

MW=429.56

Description 5. 8-Methyl-7-phenyl-2,3-dihydro-[1,4]dioxino[2,3-g]quinoline-9-carboxylic acid

1,4Benzodioxan-6-amine (10 g, 66 mmol) was dissolved in EtOH (200 ml) and benzaldehyde (6.7 ml, 66 mmol) was added. The solution was refluxed for 2 h and then 2-oxobutirric acid (6.7 g, 66 mmol) was added. The mixture was heated to reflux for additional 3 h and then cooled overnight. A solid precipitated, which was collected by suction, yielding the title compound, which may be used without further purification.

C₁₉H₁₅NO₄

MW=321.33

Description 6. 8-Methyl-7-phenyl-2,3-dihydro-[1,4]dioxino[2,3-g]quinoline-9-carboxylic acid ((S)1-cyclohexyl-ethyl)-amide

8-Methyl-7-phenyl-2,3-dihydro-[1,4]dioxino[2,3-g]quinoline-9-carboxylic acid (2.25 g, 7 mmol), prepared as in Description 5, was suspended in CH₂Cl₂ (35 ml) and oxalyl chloride (1.3 ml, 15 mmol) was added dropwise at room temperature. After adding two drops of DMF, stirring was continued for 1 h. Additional oxalyl chloride (0.7 ml, 7 mmol) was added to the solution and after 1 h the organic solvent was evaporated to dryness. The solid was dissolved in CH₂Cl₂ and added dropwise to a suspension of (S)-1-cyclohexylethylamine (2.15 ml, 14 mmol) and K₂CO₃ (2.9 g, 21 mmol) in CH₂Cl₂ (30 ml). The reaction was refluxed for 24 h, cool to room temperature and the solvent evaporated to dryness. The crude solid was dissolved in AcOEt and washed with NaOH, HCl, and brine. The organic phase was dried on Na₂SO₄, filtered and evaporated to dryness obtaining a crude residue that, when triturated with Et₂O, gave the title compound as a solid.

C₂₇H₃₀N₂O₃

MW=430.55

MP=217-219° C.

Description 7. 7-Methyl-6-phenyl-[1,3]dioxolo[4,5-g]quinoline-8-carboxyllc acid

3,4-Methylenedioxyaniline (20.16 g, 147 mmol) was dissolved in EtOH (300 ml) and both benzaldehyde (14.3 ml, 147 mmol) and 2-oxobutirric acid (15 g, 147 mmol) were added. The solution was stirred at room temperature for three days. A solid was formed which was collected by filtration and dissolved in NaOH 1 M. The solution was washed with Et₂O and acidification of the aqueous phase a solid precipitated. The solid was filtered by suction and dried in vacuum oven to yield the title compound as a solid.

C₁₈H₁₃NO₄

MW=307.30

MP=>300° C.

Description 8. 7-Methyl-6phenyl-[1,3]dioxolo[4,5-g]quinoline-8-carboxylic acid ((S)-1-cyclohexyl-ethyl)-amide

7-Methyl-6-phenyl-[1,3]dioxolo[4,5-g]quinoline-8-carboxylic acid (10 g, 32.5 mmol), prepared as in Description 7, was suspended in CH₂Cl₂ (200 ml) and cooled to 0-5° C. Oxalyl chloride (5.8 ml, 65 mmol) was added dropwise under stirring in 15 min. After adding few drops of DMF, the mixture was allowed to warm to room temperature and left for 3 h. The organic solvent was evaporated to dryness. The crude residue was dissolved with CH₂Cl₂ and added dropwise to a stirred suspension of (S)-1-cyclohexylethylamine (5.8 ml, 39.05 mmol) and K₂CO₃ (9 g) in CH₂Cl₂ (150 ml). The solid was filtered and the organic solvent was evaporated to dryness. The crude residue was purified by flash chromatography (eluent hexane:AcOEt 6:4) to afford the title compound as a solid.

C₂₆H₂₈N₂O₃

MW=416.52

Description 9. (3-Amino-phenyl)-methanol

The suspension of LiAlH₄ (10 g, 263 mmol) in dry THF (500 ml) was cooled to 0° C. and a solution of m-aminobenzoic acid (17.83 g, 130 mmol) in dry TBF (500 ml) was slowly added dropwise maintaining the internal temperature as constant as possible and using mechanical stirring. After the end of the addition, the suspension was refluxed for three days. H₂O (10 ml) was carefully added followed by 15% NaOH (30 ml) and H₂O (10 ml). The suspension was filtered and the organic phase was dried over Na₂SO₄, filtered and evaporated to dryness to afford the title compound, which may be used without further purification.

C₇H₉NO

MW=123.15

Description 10. 7-Hydroxymethyl-3-methyl-2-phenyl-quinoline-4-carboxylic acid

To a solution of crude (3-amino-phenyl)-methanol (18 g), prepared as in Description 9, in EtOH (400 ml) benzaldehyde (14.2 ml, 140 mmol) was added. The solution was refluxed for 2 h and then 2-oxobutirric acid (14.3 g, 140 mmol) was added. The mixture was refluxed for 3 h. The reaction was allowed to cool overnight to room temperature and the resulting precipitate was collected by filtration to afford the title compound.

C₁₈H₁₅NO₃

MW=293.32

Description 11. 7-Hydroxymethyl-3-methyl-2-phenyl-quinoline-4-carboxylic acid ((S)-1-cyclohexyl-ethyl)-amide

7-Hydroxymethyl-3-methyl-2-phenyl-quinoline-4-carboxylic acid (6,75 g, 23 mmol), prepared as in Description 10, was suspended in TBF:CH₂Cl₂ mixture (300 ml) and triethylamine (12.8 ml, 92 mmol) and HBTU (8.75 g, 23 mmol) were added. After heating the solution to 40° C. until it becomes clear (about 30 min), (S)-1-cyclohexylethylamine (6.5 ml, 46 mmol) was added and the solution kept at 40° C. for 3 h. The reaction was allowed to cool overnight to room temperature. The organic solvent was evaporated to dryness and the resulting crude residue was dissolved in AcOEt, washed with water, HCl (1 M), NaOH (1 M) and then with water. The organic phase was dried over Na₂SO₄, filtered and evaporated to dryness obtaining an oil that, when treated with Et₂O, yielded the title compound as a solid.

C₂₅H₂₈N₂O₂

MW=388.51

MP=160-161° C.

Description 12. 4-((S)-1-Cyclohexyl-ethylcarbamoyl)-3-methyl-2-phenyl-quinoline-7-carboxylic acid

A solution of 7-hydroxymethyl-3-methyl-2-phenyl-quinoline-4-carboxylic acid ((S)-1-cyclohexyl-ethyl)-amide (5 g, 12.5 mmol), prepared as in Description 11, in THF (1.25 L) was added dropwise aqueous 0.2 M KOH (2.5 L), containing K₂S₂O₈ (20.27 g, 75 mmol) and RuCl₃ (881 mg, 4.25 mmol). The resulting mixture was stirred at room temperature for 1.5 h. THF was then removed and the remaining aqueous solution washed with Et₂O. The aqueous phase was acidified with 1M HCl and extracted with AcOEt. The organic phase was then dried over Na₂SO₄, filtered and evaporated to dryness to afford the crude product. The crude product was triturated with Et₂O to afford the title compound.

C₂₆H₂₈N₂O₃

MW=416.52

MP=70° C.

Description 13. 5-[1,4′]Bipiperidinyl-1′-yl-1-phenyl-pentan-1-one

Mesyl chloride (2.5 ml, 32 mmol) in DCM (10 ml) was added dropwise at 0° C. to a solution of 5-hydroxy-1-phenyl-pentan-1-one (5 g, 28 mmol, CAS[1011-62-7]), prepared as in Description 12, and Et₃N (5 ml, 36 mmol) in DCM (65 ml). The reaction was left 30 min at 0° C. and then 1 h at room temperature. The organic layer was washed with H₂O, dried over Na₂SO₄ and then evaporated to dryness. The crude mesylate was dissolved in DMF (20 ml) and 4-piperidinopiperidine (4.7 g, 28 mmol) and Et₃N (3.9 ml, 28 mmol) were added. The reaction was stirred at 60° C. for 18 h. The organic solvent was removed under reduced pressure and the crude compound was purified by column chromatography (AcOEt/ MeOH 7:3 and then 1:1) to afford the title compound as an oil.

Description 14. 3-(3-[1,4′]Bipiperidinyl-1′-yl-propyl)-2-phenyl-quinoline-4-carboxylic acid

A suspension of isatine (2.24 g, 15.2 mmol) and KOH (3.42 g, 61 mmol) in EtOH (200 ml) was stirred for 1.5 h at room temperature. 5-[1,4′]Bipiperidinyl-1′-yl-1-phenyl-pentan-1-one (5 g, 15.2 mmol), prepared as in Description 13, was added and the reaction was refluxed for 6 days. The organic solvent was removed under vacuum and the residue was dissolved in H₂O, acidified with HCl to pH 1 and then extracted with DCM. The organic phase was dried over Na₂SO₄ and evaporated to dryness to afford the title compound.

Description 15: 6,7-Dimethoxy-3-methyl-2-phenyl-quinoline-4-carboxylic acid ((S)-1-cyclohexyl-ethyl)-amide

This compound was prepared starting from 3,4-dimethoxyaniline and following the procedure described in Description 7-8.

Description 16: 3-Bromomethyl-6,7-dimethoxy-2-phenyl-quinoline-4-carboxylic acid ((S)-1-cyclohexyl-ethyl)-amide

6,7-Dimethoxy-3-methyl-2-phenyl-quinoline-4-carboxylic acid ((S)-1-cyclohexyl-ethyl)-amide (0.7 g, 1.62 mmol), prepared as in Description 15, was suspended in CH₃CN (40 ml) and warmed to 50° C. N-Bromosuccinimide (1.44 g, 8.1 mmol) and dibenzoylperoxide (30 mg) were added. The reaction was refluxed for 4 h. The organic solvent was removed under reduced pressure to afford the title compound which may be used without further purification.

Description 17. 1-(4-Methoxy-cyclohexyl)-ethylamine hydrochloride

(S)-1-(4-Methoxyphenyl)ethylamine (1 g, 6 mmol), dissolved in EtOH (20 ml) containing acetic acid (1 ml), was hydrogenated over Rh/Al₂O₃ at 55 psi for 16 h at room temperature. The catalyst was filtered off and the organic solvent was evaporated to dryness. The residue was dissolved in EtOAc, acidified with HCl and evaporated to dryness. This operation was repeated several times to afford the tile compound which may be without further purification.

Description 18: 1-(2-Fluoro-phenyl)-propylamine

2-Fluoropropiophenone (1.5 g, 9.86 mmol) and O-methylhydroxylamina hydrochloride (1.5 g, 20 mmol) were dissolved in EtOH/H₂O 3:1 (15 ml). 32% NaOH (2.1 ml) was added dropwise. The reaction was stirred overnight at room temperature and then refluxed for 4 h. The organic solvent was removed under reduced pressure and the residue was dissolved in Et₂O, washed with water, dried over Na₂SO₄, filtered and evaporated to dryness obtaining 1.35 g of the crude 0-methyl-oxime intermediate. This compound was dissolved in anhydrous THF (15 ml) and 1M BH₃ in THF (7 ml) was added at 0° C. The reaction was refluxed for 3h. After cooling to 0° C., H20 (10 ml) and 20% KOH (10 ml) were added dropwise. The reaction was refluxed for about 2 h. The organic solvent was removed under reduced pressure and the residue was dissolved in Et₂O and extracted with 2M HCl. The aqueous phase was basified with 1M NaOH and extracted with Et₂O. The organic phase was dried over Na₂SO₄, filtered and evaporated to dryness to afford the title compound, which may be used without further purification.

Description 19: 7-Methyl4-phenyl-[1,3]dioxolo[4,5-g]quinoline-8-carboxylic acid methyl ester

7-Methyl-6-phenyl-[1,3]dioxolo[4,5-g]quinoline-8-carboxylic acid (10 g, 32.5 mmol), prepared as in Description 7, was suspended in CH₂Cl₂ (200 ml) and cooled to 0-5° C. Oxalyl chloride (5.8 ml, 65 mmol) was added dropwise under stirring in 15 min. After adding few drops of DMF, the mixture was allowed to warm to room temperature and left for 3 h. The organic solvent was evaporated to dryness. The crude residue was dissolved in MeOH and refluxed for 4 h. The organic solvent is evaporated to dryness to afford the title compound.

Description 20: 7-Bromomethyl-6-phenyl-[1,3]dioxolo[4,5-g]quinoline-8-carboxylic acid methyl ester

To a solution of 7-methyl-6phenyl-[1,3]dioxolo[4,5-g]quinoline-8-carboxylic acid methyl ester (8.27 g, 25.74 mmol), prepared as in Description 19, in CH₃CN (400 ml), N-bromosuccimide (18.32 g, 102.25 mmol) and benzoylperoxide (0.3 g) were added. The reaction was carefully heated under reflux for 5 h. N-bromosuccimide (4.6 g, 25.5 mmol) and benzoylperoxide (0.3 g) were added and the reaction was refluxed overnight. The organic solvent was removed under reduced pressure and the crude reaction product was used in the next step without further purification.

Description 21: 7-[1,4′]Bipiperidinyl-1′-ylmethyl-6-phenyl-[1,3]dioxolo[4,5-g]quinoline-8-carboxylic acid methyl ester

The crude 7-bromomethyl-6-phenyl-[1,3]dioxolo[4,5-g]quinoline-8-carboxylic acid methyl ester, prepared as in Description 20, was dissolved in CH₃CN (100 ml) and added dropwise to a suspension of K₂CO₃ (14.23 g) and 4-piperidinopiperidine (9.6 g, 588 mmol) in CH₃CN (300 ml). The reaction was refluxed for 4 h. The organic solvent was removed under reduced pressure and the residue was re-dissolved in EtOAc, washed with 0.5N HCl, dried over Na₂SO₄, filtered and evaporated to dryness. The solid is triturated with Et₂O to give, after filtration, the title compound together with other brominated quinoline derivatives.

Compound prepared as above dissolved in AcOEt/THF 1:1 (125 ml), was hydrogenated over 10% Pd/C (480 mg) at 6 psi for 70 h at room temperature. The catalyst was filtered off and the organic solvent was evaporated to dryness to afford the title compound.

Description 22: 7-[1,4′]Bipiperidinyl-1′-ylmethyl-6-phenyl-[1,3]dioxolo[4,5-g]quinoline-8-carboxylic acid hydrochloride

7-[1,4′]Bipiperidinyl-1′-ylmethyl-6-phenyl-[1,3]dioxolo[4,5-g]quinoline-8-carboxylic acid methyl ester (0.5 g, 1 mmol) was dissolved in 6M HCl and refluxed for 1.5 h. The reaction was evaporated to dryness, dissolved in CH₃CN and evaporated to dryness. This operation was repeated several times to afford the title compound as a solid.

Example 1: 8-[1,4′]Bipiperidinyl-1′-ylmethyl-7-phenyl-2,3-dihydro-[1,4]dioxino[2,3-g]quinoline-9-carboxylic acid ((S)-1-cyclohexyl-ethyl)-amide

8-Methyl-7-phenyl-2,3-dihydro-[1,4]dioxino[2,3-g]quinoline-9-carboxylic acid ((S)-1-cyclohexyl-ethyl)-amide (1.07 g, 2.5 mmol), prepared as in Description 6, was suspended in CCl4 (40 ml) and NBS (0.89 g, 5 mmol) was added. The mixture was heated to reflux and 10 mg of benzoylperoxide were added. After 1 hour the mixture had cleared and all the starting material had been consumed. A 10% of dibrominated compound was also detected. The solvent was evaporated and the residue was taken up with CH₃CN (50 ml). The solution was added dropwise to a suspension of 4-piperidinopiperidine (0.84 g, 5 mmol) and K₂CO₃ (0.7 g, 5 mmol) in CH₃CN (50 ml). The reaction was refluxed for 1 h. The mixture was allowed to cool to room temperature, filtered and the organic solvent evaporated to dryness. The oil was purified by column cromatography (eluent CH₂Cl₂/MeOH/NH₃ 95/5/0.5) to afford the title compound as a solid.

Example 2: 7-[1,4′]Bipiperidinyl-1′-ylmethyl-6-phenyl-[1,3]dioxolo[4,5g]quinoline-9-carboxylic acid ((S)-1-cyclohexyl-ethyl)-amide

7-Methyl-6-phenyl-[1,3]dioxolo[4,5-g]quinoline-8-carboxylic acid ((S)-1-cyclohexyl-ethyl)-amide (1 g, 2.4 mmol), prepared as in Description 8, was suspended in CCl₄ (30 ml). After adding NBS (1.71 g, 9.60 mmol) and benzoyl peroxide (80 mg), the suspension was refluxed for 3.5 h. A major product was observed along with a minor one, identified as a dibromo-derivative. The reaction was allowed to cool to room temperature and the solvent was evaporated to dryness. The residue was treated with CH₂Cl₂ and the solid filtered. The resulting organic solution was added dropwise to a suspension of 4-piperidinopiperidine (880 mg, 4.8 mmol) and K₂CO₃ (1.23 g) in CH₂Cl₂ (25 ml). After stirring for 3 h, the mixture was filtered and evaporated to dryness. The crude residue was treated with AcOEt; filtering the insoluble solid. The organic phase was washed with water, dried over Na₂SO₄, filtered and evaporated to dryness. The crude solid was then purified by flash chromatography (eluent CH₂Cl₂:MeOH:NH₃ 98:2:0.5), yielding a solid, which was identified as being a mixture of the title compound and a related monobrominated derivative.

The mixture (900 mg), obtained as described above, was dissolved in EtOH and hydrogenated at room temperature under a 10 psi H₂ pressure, in presence of Pd/BaSO₄ (130 mg) and K₂CO₃ (180 mg) as catalyst. After 18 h the reaction was complete; the catalyst was filtered and the organic solvent evaporated to dryness. The resulting residue was purified by flash chromatography (eluent AcOEt:MeOH:NH₃ 95:5:0.5) to afford the title compound as a powder.

Example 3: 3-[1,4′]Bipiperidinyl-1′-ylmethyl-4-((S)-1-cyclohexyl-ethylcarbamoyl)-2-phenyl-quinoline-7-carboxylic acid

4-((S)-1-Cyclohexyl-ethylcarbamoyl)-3-methyl-2-phenyl-quinoline-7-carboxylic acid (3.54 g, 8.5 mmol), prepared as in Description 12, was dissolved in 1,2-dichloroethane (200 ml) and NBS (6 g, 34 mmol) was added. The solution was heated to reflux before adding benzoylperoxide (100 mg). The suspension was refluxed for 3 h and then the organic solvent was evaporated to dryness. The crude residue was dissolved in CH₃CN and the solution was added dropwise to a solution of 4-piperidinopiperidine (5.7 g, 34 mmol) in CH₃CN. The mixture was refluxed for 30 min. The precipitate was removed by filtration and the organic solvent was evaporated to dryness. The crude residue was purified by column chromatography (eluent CH₂Cl₂/MeOH/NH₃ 80/20/2), to afford the title compound.

Examples 4-67 and 71: General procedure for synthesis of 3-{[4-(1-piperidinyl)-1-piperidinyl]methyl}-2-phenylquinoline-4-carboxamides

135 mg (˜0.23 mmol) of 3-[1,4′]bipiperidinyl-1′-ylmethyl-2-phenyl-quinoline-4-carboxylic acid, prepared as in Description 4, were dissolved in CH₂Cl₂ (4 ml) and 270 mg of N-dicyclohexylcarbodiimide, N-methyl polystyrene resin (loading 1.69 mmol/g) were added. One equivalent of the amine, dissolved in CH₂Cl₂ (0.5 ml), was added to the mixture. If the amine was a hydrochloride salt, one equivalent of tetramethylguanidine was also added to the reaction mixture. In case of poor solubility of the amine, DMF (up to 2 ml) was added. After stirring for 3 days, the reaction was complete. The resin was filtered and the obtained solution evaporated. The resulting solid was then purified by flash chromatography (usual eluent CH₂Cl₂:MeOH:NH₃ 95:5:0.3) on a Biotage QUAD3 multi-column apparatus. The fractions containing the product were evaporated to afford the tile compound.

Example 68: 3-(3-[1,4′]Bipiperidinyl-1′-yl-propyl)-2-phenyl-quinoline-4-carboxylic acid ((S)-1-cyclohexyl-ethyl)-amide

3-(3-[1,4′]Bipiperidinyl-1′-yl-propyl)-2-phenyl-quinoline-4-carboxylic acid (0.29 g, 0.64 mmol), prepared as in Description 14, was dissolved in ThF (7 ml) HBTU (0.32 g, 0.83 mmol) and Et₃N (0.29 ml, 2.11 mmol) were added at room temperature. The reaction was stirred for 2 h. (S)-1-Cyclohexyl-ethylamine (0.14 ml, 0.96 mmol), dissolved in THF (4 ml) was added dropwise and the reaction was stirred for 20 h at room temperature. The organic solvent was evaporated to dryness, and the residue was dissolved in AcOEt, washed with brine, dried over Na₂SO₄, filtered and evaporated to dryness. The residue was purified by column chromatography (eluent CH₂Cl₂/MeOH/NH₄OH 95:5:0.3) to afford the title compound as a solid.

Example 69: 3-[1,4′]Bipiperidinyl-1′-ylmethyl-2-phenylquinoline-4-carboxylic acid [1-(4-methoxy-cyclohexyl)-ethyl]-amide

The compound was prepared following the procedure of Example 4-67 starting from the compounds prepared in Description 4 and in Description 17.

Example 70: 3-[1,4′]Bipiperidinyl-1′-ylmethyl-6,7-dimethoxy-2-phenyl-quinoline-4-carboxylic acid ((S)-1-cyclohexyl-ethyl)-amide

3-Bromomethyl-6,7-dimethoxy-2-phenyl-quinoline-4-carboxylic acid ((S)-1-cyclohexyl-ethyl)-amide (0.3 g), prepared as in Description 16, dissolved in CH₃CN/CH₂Cl₂4:1 (15 ml), was added dropwise to a suspension of K₂CO₃ (0.122 g, 0.88 mmol) and 4-piperidinopiperidine (0.081 g, 0.44 mmol) in CH₃CN (30 ml). The reaction was refluxed for 8 h. The organic solved was removed under reduced pressure and the residue was re-dissolved in CH₂Cl₂, washed with H₂O, dried over Na₂SO₄, filtered and evaporated to dryness. The compound was purified by column chromatography (eluent AcOEt/MeOH/NH4OH 95:5:0.3) to afford a mixture of the title compound and of the corresponding mono- and di-brominated quinoline derivative(s). This mixture, dissolved in EtOH (100 ml) with Pd/BaSO₄ (10 mg) and K₂CO₃ (41 mg, 0.3 mmol), was hydrogenated at 10 psi for 12 h. The reaction was filtered and the solvent was evaporated to dryness to afford a crude residue that was purified by column chromatography to obtain the title compound as a solid.

Example 72: 3-[1,4′]Bipiperidinyl-1′-ylmethyl-2-phenyl-quinoline-4-carboxylic acid [1-(2-fluoro-phenyl)-propyl]-amide

The compound was prepared following the procedure of Example 4-67 starting from the compounds prepared in Description 4 and in Description 18.

Example 73: 7-[1,4′]Bipiperidinyl-1′-ylmethyl-6-phenyl-[1,3]dioxolo[4,5-g]quino line-8-carboxylic acid ((S)-1-phenyl-propyl)-amide

The compound of Example 73 was prepared following the procedure of Example 4-67 starting from (S)-1-phenyl-propylamine and the compound prepared in Description 22.

TABLE 1
(Examples)

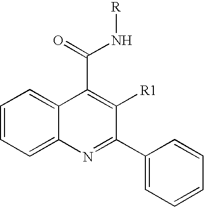

| Example | R | R1 | Molecular formula |
|---|---|---|---|
| 1 | 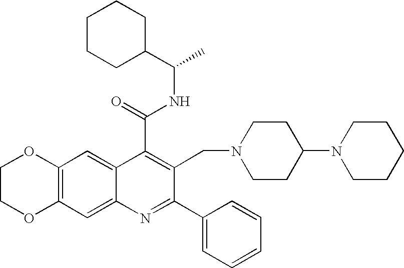 | | C₃₇H₄₈N₄O₃ |
| 2 | 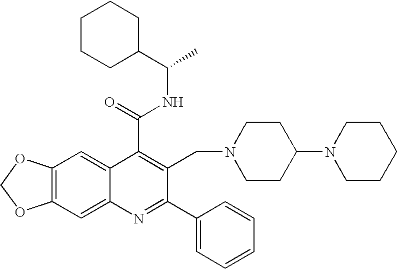 | | C₃₆H₄₆N₄O₃ |
| 3 | 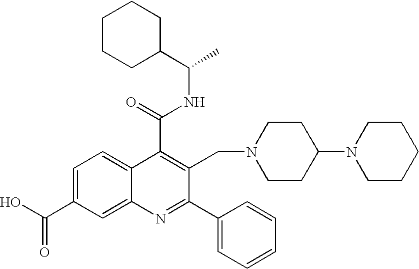 | | C₃₆H₄₆N₄O₃ |
| 4 | 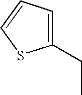 | 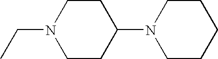 | C₃₂H₃₆N₄OS |
| 5 | 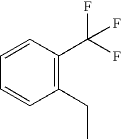 | 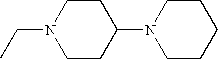 | C₃₅H₃₇F₃N₄O |
| 6 | 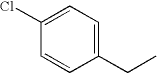 | 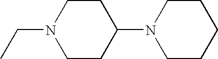 | C₃₄H₃₇ClN₄O |
| 7 | 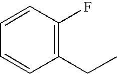 | 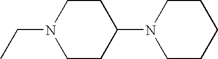 | C₃₄H₃₇FN₄O (C₃₄H₃₇FN₄O.C₂HF₃O₂) |
| 8 | 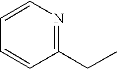 | 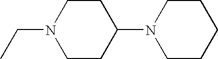 | C₃₃H₃₇N₅O |
| 9 | 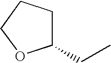 | 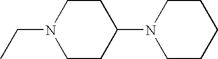 | C₃₂H₄₀N₄O₂ |
| 10 | 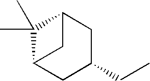 | 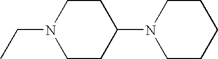 | C₃₇H₄₈N₄O |
| 11 | 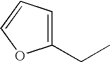 | 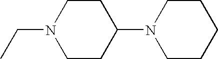 | C₃₂H₃₆N₄O₂(C₃₂H₃₆N₄O₂.C₂HF₃O₂) |
| 12 | 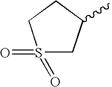 | 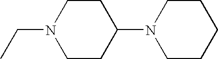 | C₃₁H₃₈N₄O₃S |
| 13 | 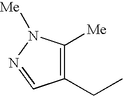 | 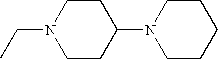 | C₃₃H₄₀N₆O |
| 14 | 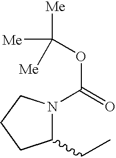 | 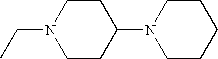 | C₃₇H₄₉N₅O₃ |
| 15 | 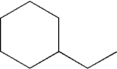 | 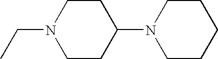 | C₃₄H₄₄N₄O |
| 16 | 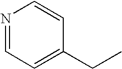 | 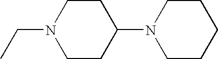 | C₃₃H₃₇N₅O |
| 17 | 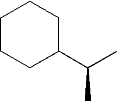 | 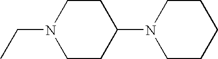 | C₃₅H₄₆N₄O |
| 18 | 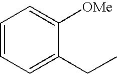 | 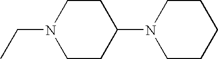 | C₃₅H₄₀N₄O₂ |
| 19 | 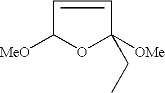 | 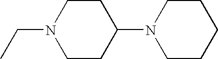 | C₃₄H₄₂N₄O₄ |
| 20 | 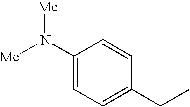 | 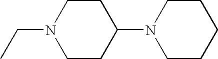 | C₃₆H₄₃N₅O |
| 21 | 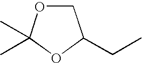 | 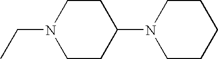 | C₃₃H₄₂N₄O₃ |
| 22 | 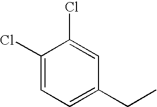 | 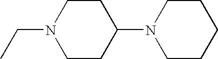 | C₃₄H₃₆Cl₂N₄O |
| 23 | 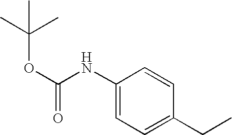 | 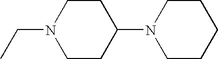 | C₃₉H₄₇N₅O₃ |
| 24 | 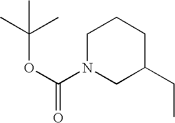 | 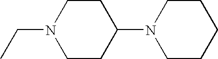 | C₃₈H₅₁N₅O₃ |
| 25 | 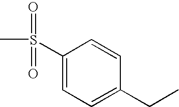 | 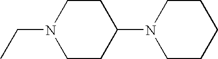 | C₃₅H₄₀N₄O₃S |
| 26 | 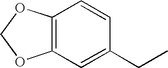 | 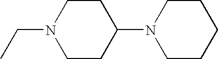 | C₃₅H₃₈N₄O₃ |
| 27 | 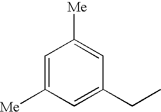 | 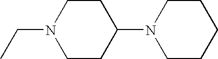 | C₃₆H₄₂N₄O |
| 28 | 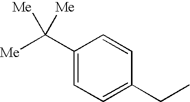 | 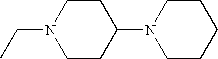 | C₃₈H₄₆N₄O |
| 29 | 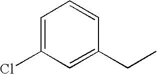 | 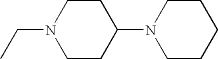 | C₃₄H₃₇ClN₄O |
| 30 | 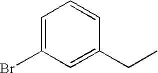 | 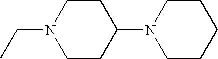 | C₃₄H₃₇BrN₄O |
| 31 | 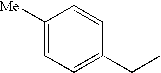 | 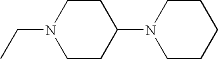 | C₃₅H₄₀N₄O |
| 32 | 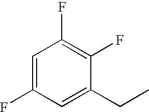 | 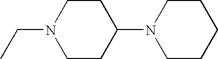 | C₃₄H₃₅F₃N₄O |
| 33 | 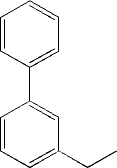 | 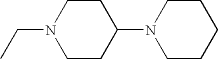 | C₄₀H₄₂N₄O |
| 34 | 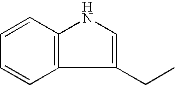 | 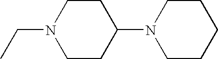 | C₃₆H₃₉N₅O |
| 35 | 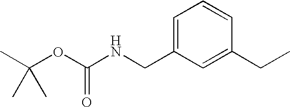 | 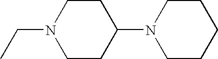 | C₄₀H₄₉N₅O₃ |
| 36 | 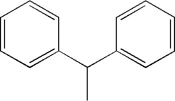 | 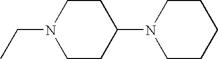 | C₄₀H₄₂N₄O |
| 37 | 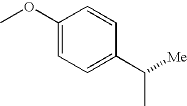 | 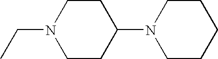 | C₃₆H₄₂N₄O₂ |
| 38 | 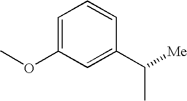 | 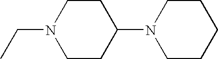 | C₃₆H₄₂N₄O₂ |
| 39 | 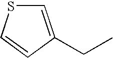 | 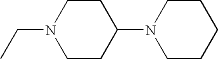 | C₃₂H₃₆N₄OS |
| 40 | 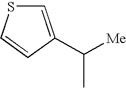 | 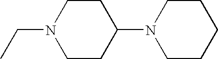 | C₃₃H₃₈N₄OS |
| 41 | 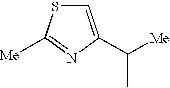 | 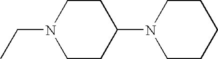 | C₃₃H₃₉N₅OS |
| 42 | 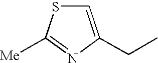 | 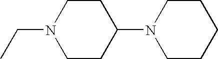 | C₃₂H₃₇N₅OS |
| 43 | 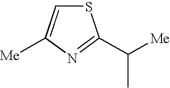 | 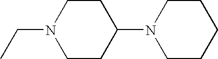 | C₃₃H₃₉N₅OS |
| 44 | 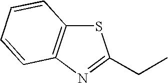 | 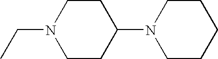 | C₃₅H₃₇N₅OS |
| 45 | 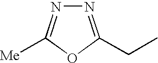 | 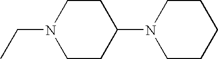 | C₃₁H₃₆N₆O₂ |
| 46 | 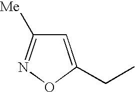 | 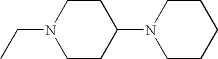 | C₃₂H₃₇N₅O₂ |
| 47 | 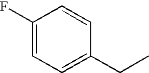 | 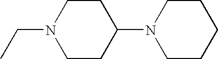 | C₃₄H₃₇FN₄O |
| 48 | 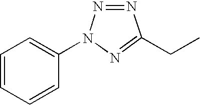 | 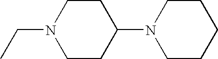 | C₃₆H₃₉N₇O |
| 49 | 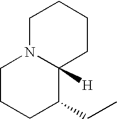 | 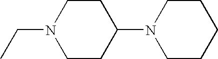 | C₃₇H₄₉N₅O |
| 50 | 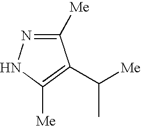 | 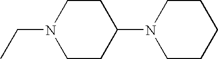 | C₃₄H₄₂N₆O |
| 51 | 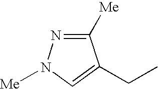 | 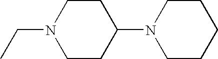 | C₃₃H₄₀N₆O |
| 52 | 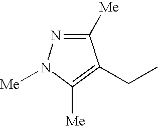 | 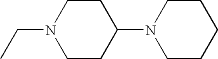 | C₃₄H₄₂N₆O |
| 53 | 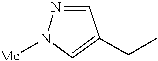 | 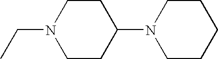 | C₃₂H₃₈N₆O |
| 54 | 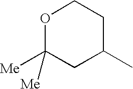 | 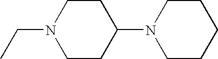 | C₃₄H₄₄N₄O₂ |
| 55 | 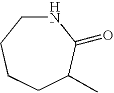 | 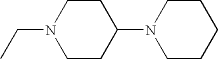 | C₃₃H₄₁N₅O₂ |
| 56 | 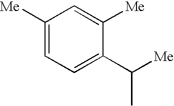 | 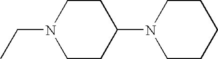 | C₃₇H₄₄N₄O |
| 57 | 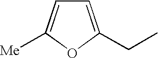 | 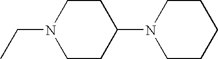 | C₃₃H₃₈N₄O₂ |
| 58 | 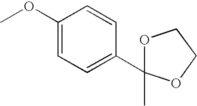 | 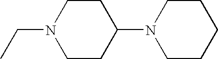 | C₃₈H₄₄N₄O₄ |
| 59 | 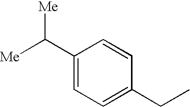 | 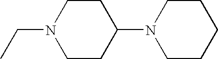 | C₃₇H₄₄N₄O |
| 60 | 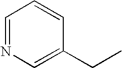 | 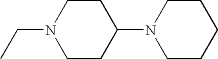 | C₃₃H₃₇N₅O |
| 61 | 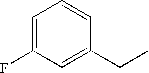 | 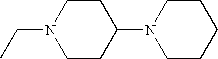 | C₃₄H₃₇FN₄O |
| 62 | 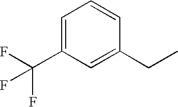 | 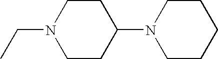 | C₃₅H₃₇F₃N₄O |
| 63 | 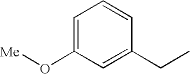 | 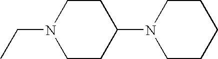 | C₃₅H₄₀N₄O₂ |
| 64 | 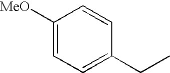 | 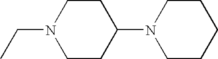 | C₃₅H₄₀N₄O₂ |
| 65 | 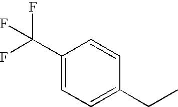 | 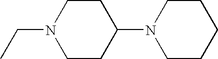 | C₃₅H₃₇F₃N₄O |
| 66 | 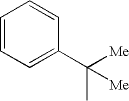 | 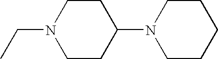 | C₃₅H₄₂N₄O |
| 67 | 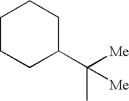 | 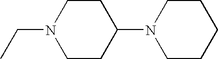 | C₃₆H₄₈N₄O |
| 68 | 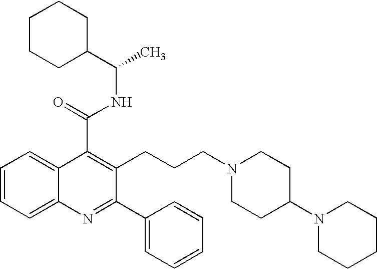 | | C₃₇H₅₀N₄O |
| 69 | 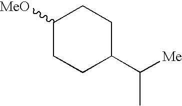 | 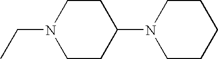 | C₃₆H₄₈N₄O₂ |
| 70 | 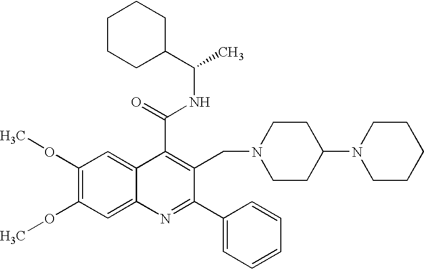 | | C₃₇H₅₀N₄O₃ |
| 71 | 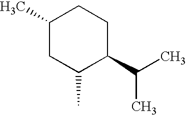 | 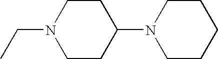 | C₃₇H₅₀N₄O |
| 72 | 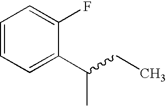 | 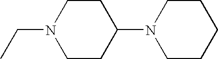 | C₃₆H₄₁FN₄O |

| | | |
|---|---|---|
| 73 | 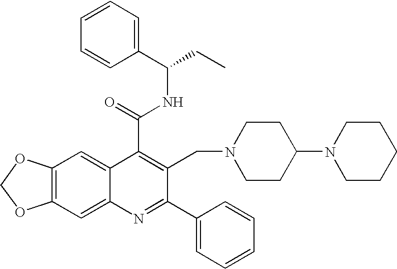 | C₃₇H₄₂N₄O₃ |

TABLE 2
¹H NMR and/or mass spectroscopy data of compounds of Examples of Table 1

| Ex | ¹H NMR(Solvent)ppm and/or MS |
|---|---|
| 1 | ¹H NMR(DMSO, 343K)δ: 8.22(d br, 1H); 7.51(m, 2H); 7.47-7.39(m, 3H); 7.37 |
| | (s, 1H); 7.20(s, 1H); 4.39(s, 4H); 4.01(m, 1H); 3.50(s, 2H); 2.52(m, 2H); 2.36(m, |
| | 4H); 1.99(m, 1H); 1.88-1.60(m, 7H); 1.53-1.05(m, 16H); 1.18(d, 3H). |
| | EI; TSQ 700; source 180° C.; 70V; 200uA: 596(M+.); 430; 277; 167; 124 |
| 2 | ¹H NMR(DMSO, 343K)δ: 8.24(d br, 1H); 7.51(m, 2H); 7.47-7.39(m, 3H); 7.31 |
| | (s, 1H); 7.09(s, 1H); 6.19(d, 2H); 4.01(m, 1H); 3.52(s, 2H); 2.54(m, 2H); 2.36(m, |
| | 4H); 2.01(m, 1H); 1.86-1.60(m, 7H); 1.53-1.05(m, 16H); 1.18(d, 3H). |
| | ESI POS; AQA; solvent: MeOH/spray 3kV/skimmer: 20V/probe 135° C.: 583 |
| | (MH+) |
| 3 | ¹H NMR(DMSO, 343K, as sodium salt): 8.41(d, 1H); 8.24(d br, 1H); 8.09(dd, |
| | 1H); 7.71(d, 1H); 7.56(m, 2H); 7.49-7.39(m, 3H); 4.03(m, 1H); 3.54(s, 2H); 2.44 |
| | (m, 2H); 2.35(m, 4H); 1.96(m, 1H); 1.88-1.60(m, 7H); 1.53-1.05(m, 16H); 1.19(d, |
| | 3H). |
| | ESI POS; AQA; solvent: MeOH/spray 3kV/skimmer: 20V/probe 135° C.: 584 |
| | (MH+) |
| 4 | ESI POS; AQA; solvent: MeOH/spray 3kV/skimmer: 20V/probe 135° C.: 525 |
| | (MH+) |
| 5 | ESI POS; AQA; solvent: MeOH/spray 3kV/skimmer: 20V/probe 135° C.: 587 |
| | (MH+) |
| 6 | ESI POS; AQA; solvent: MeOH/spray 3kV/skimmer: 20V/probe 135° C.: 553 |
| | (MH+) |
| 7 | ESI POS; AQA; solvent: MeOH/spray 3kV/skimmer: 20V/probe 135° C.: 537 |
| | (MH+) |
| 8 | ESI POS; AQA; solvent: MeOH/spray 3kV/skimmer: 20V/probe 135° C.: 520 |
| | (MH+) |
| 9 | ESI POS; AQA; solvent: MeOH/spray 3kV/skimmer: 20V/probe 135° C.: 513 |
| | (MH+) |
| 10 | ESI POS; AQA; solvent: MeOH/spray 3kV/skimmer: 20V/probe 135° C.: 565 |
| | (MH+) |
| 11 | ESI POS; AQA; solvent: MeOH/spray 3kV/skimmer: 20V/probe 135° C.: 509 |
| | (MH+) |
| 12 | ESI POS; AQA; solvent: MeOH/spray 3kV/skimmer: 20V/probe 135° C.: 547 |
| | (MH+) |
| 13 | ESI POS; AQA; solvent: MeOH/spray 3kV/skimmer: 20V/probe 135° C.: 537 |
| | (MH+) |
| 14 | ESI POS; AQA; solvent: MeOH/spray 3kV/skimmer: 20V/probe 135° C.: 612 |
| | (MH+) |
| 15 | ESI POS; AQA; solvent: MeOH/spray 3kV/skimmer: 20V/probe 135° C.: 525 |
| | (MH+) |
| 16 | ESI POS; AQA; solvent: MeOH/spray 3kV/skimmer: 20V/probe 135° C.: 520 |
| | (MH+) |
| 17 | ESI POS; AQA; solvent: MeOH/spray 3kV/skimmer: 20V/probe 135° C.: 539 |
| | (MH+) |
| 18 | ESI POS; AQA; solvent: MeOH/spray 3kV/skimmer: 20V/probe 135° C.: 549 |
| | (MH+) |
| 19 | ESI POS; AQA; solvent: MeOH/spray 3kV/skimmer: 20V/probe 135° C.: 571 |
| | (MH+) |
| 20 | ESI POS; AQA; solvent: MeOH/spray 3kV/skimmer: 20V/probe 135° C.: 562 |
| | (MH+) |
| 21 | ESI POS; AQA; solvent: MeOH/spray 3kV/skimmer: 20V/probe 135° C.: 543 |
| | (MH+) |
| 22 | ESI POS; AQA; solvent: MeOH/spray 3kV/skimmer: 20V/probe 135° C.: 587 |
| | (MH+) |
| 23 | ESI POS; AQA; solvent: MeOH/spray 3kV/skimmer: 20V/probe 135° C.: 634 |
| | (MH+) |
| 24 | ESI POS; AQA; solvent: MeOH/spray 3kV/skimmer: 20V/probe 135° C.: 626 |
| | (MH+) |
| 25 | ESI POS; AQA; solvent: MeOH/spray 3kV/skimmer: 20V/probe 135° C.: 597 |
| | (MH+) |
| 26 | ESI POS; AQA; solvent: MeOH/spray 3kV/skimmer: 20V/probe 135° C.: 563 |
| | (MH+) |
| 27 | ESI POS; AQA; solvent: MeOH/spray 3kV/skimmer: 20V/probe 135° C.: 547 |
| | (MH+) |
| 28 | ESI POS; AQA; solvent: MeOH/spray 3kV/skimmer: 20V/probe 135° C.: 575 |
| | (MH+) |
| 29 | ESI POS; AQA; solvent: MeOH/spray 3kV/skimmer: 20V/probe 135° C.: 553 |
| | (MH+) |
| 30 | ESI POS; AQA; solvent: MeOH/spray 3kV/skimmer: 20V/probe 135° C.: 598 |
| | (MH+) |
| 31 | ESI POS; AQA; solvent: MeOH/spray 3kV/skimmer: 20V/probe 135° C.: 533 |
| | (MH+) |
| 32 | ESI POS; AQA; solvent: MeOH/spray 3kV/skimmer: 20V/probe 135° C.: 573 |
| | (MH+) |
| 33 | ESI POS; AQA; solvent: MeOH/spray 3kV/skimmer: 20V/probe 135° C.: 595 |
| | (MH+) |
| 34 | ESI POS; AQA; solvent: MeOH/spray 3kV/skimmer: 20V/probe 135° C.: 558 |
| | (MH+) |
| 35 | ESI POS; AQA; solvent: MeOH/spray 3kV/skimmer: 20V/probe 135° C.: 648 |
| | (MH+) |
| 36 | ESI POS; AQA; solvent: MeOH/spray 3kV/skimmer: 20V/probe 135° C.: 595 |
| | (MH+) |
| 37 | ESI POS; AQA; solvent: MeOH/spray 3kV/skimmer: 20V/probe 135° C.: 563 |
| | (MH+) |
| 38 | ESI POS; AQA; solvent: MeOH/spray 3kV/skimmer: 20V/probe 135° C.: 563 |
| | (MH+) |
| 39 | ESI POS; AQA; solvent: MeOH/spray 3kV/skimmer: 20V/probe 135° C.: 525 |
| | (MH+) |
| 40 | ESI POS; AQA; solvent: MeOH/spray 3kV/skimmer: 20V/probe 135° C.: 539 |
| | (MH+) |
| 41 | ESI POS; AQA; solvent: MeOH/spray 3kV/skimmer: 20V/probe 135° C.: 554 |
| | (MH+) |
| 42 | ESI POS; AQA; solvent: MeOH/spray 3kV/skimmer: 20V/probe 135° C.: 540 |
| | (MH+) |
| 43 | ESI POS; AQA; solvent: MeOH/spray 3kV/skimmer: 20V/probe 135° C.: 554 |
| | (MH+) |
| 44 | ESI POS; AQA; solvent: MeOH/spray 3kV/skimmer: 20V/probe 135° C.: 576 |
| | (MH+) |
| 45 | ESI POS; AQA; solvent: MeOH/spray 3kV/skimmer: 20V/probe 135° C.: 525 |
| | (MH+) |
| 46 | ESI POS; AQA; solvent: MeOH/spray 3kV/skimmer: 20V/probe 135° C.: 524 |
| | (MH+) |
| 47 | ESI POS; AQA; solvent: MeOH/spray 3kV/skimmer: 20V/probe 135° C.: 537 |
| | (MH+) |
| 48 | ESI POS; AQA; solvent: MeOH/spray 3kV/skimmer: 20V/probe 135° C.: 586 |
| | (MH+) |
| 49 | ESI POS; AQA; solvent: MeOH/spray 3kV/skimmer: 20V/probe 135° C.: 580 |
| | (MH+) |
| 50 | ESI POS; AQA; solvent: MeOH/spray 3kV/skimmer: 20V/probe 135° C.: 551 |
| | (MH+) |
| 51 | ESI POS; AQA; solvent: MeOH/spray 3kV/skimmer: 20V/probe 135° C.: 537 |
| | (MH+) |
| 52 | ESI POS; AQA; solvent: MeOH/spray 3kV/skimmer: 20V/probe 135° C.: 551 |
| | (MH+) |
| 53 | ESI POS; AQA; solvent: MeOH/spray 3kV/skimmer: 20V/probe 135° C.: 523 |
| | (MH+) |
| 54 | ESI POS; AQA; solvent: MeOH/spray 3kV/skimmer: 20V/probe 135° C.: 541 |
| | (MH+) |
| 55 | ESI POS; AQA; solvent: MeOH/spray 3kV/skimmer: 20V/probe 135° C.: 540 |
| | (MH+) |
| 56 | ESI POS; AQA; solvent: MeOH/spray 3kV/skimmer: 20V/probe 135° C.: 561 |
| | (MH+) |
| 57 | ESI POS; AQA; solvent: MeOH/spray 3kV/skimmer: 20V/probe 135° C.: 523 |
| | (MH+) |
| 58 | ESI POS; AQA; solvent: MeOH/spray 3kV/skimmer: 20V/probe 135° C.: 621 |
| | (MH+) |
| 59 | ESI POS; AQA; solvent: MeOH/spray 3kV/skimmer: 20V/probe 135° C.: 561 |
| | (MH+) |
| 60 | ESI POS; AQA; solvent: MeOH/spray 3kV/skimmer: 20V/probe 135° C.: 520 |
| | (MH+) |
| 61 | ESI POS; AQA; solvent: MeOH/spray 3kV/skimmer: 20V/probe 135° C.: 537 |
| | (MH+) |
| 62 | ESI POS; AQA; solvent: MeOH/spray 3kV/skimmer: 20V/probe 135° C.: 587 |
| | (MH+) |
| 63 | ESI POS; AQA; solvent: MeOH/spray 3kV/skimmer: 20V/probe 135° C.: 549 |
| | (MH+) |
| 64 | ESI POS; AQA; solvent: MeOH/spray 3kV/skimmer: 20V/probe 135° C.: 549 |
| | (MH+) |
| 65 | ESI POS; AQA; solvent: MeOH/spray 3kV/skimmer: 20V/probe 135° C.: 587 |
| | (MH+) |
| 66 | ESI POS; AQA; solvent: MeOH/spray 3kV/skimmer: 20V/probe 135° C.: 547 |
| | (MH+) |
| 67 | ESI POS; AQA; solvent: MeOH/spray 3kV/skimmer: 20V/probe 135° C.: 553 |
| | (MH+) |
| 68 | ¹H NMR(DMSO, 343K as a base)δ: 8.82(d br, 1H); 7.53-7.25(m, 11H); 6.90(s, |
| | 1H); 6.19 and 6.12(ABq, 2H); 5.05(dt; 1H); 3.45 and 3.40(Abq, 2H); 2.53-2.36(m, |
| | 8H); 2.07(m, 1H); 1.87(m, 2H); 1.65-1.34(m, 8H); 1.11(m, 2H); 0.95(t, 3H) |
| | ESI POS; AQA; solvent: MeOH/spray 3kV/skimmer: 20V/probe 135° C.: 591 |
| | (MH+) |
| 69 | ¹H NMR(DMSO, 333K)δ: 8.32(d br, 1H); 8.01(d, 1H); 7.87(d, 1H); 7.76(dd, |
| | 1H); 7.62(dd, 1H); 7.55(m, 2H); 7.50-7.39(m, 3H); 4.06(m, 1H); 3.55(s, 2H); 3.39 |
| | (m, 1H); 3.23(s, 3H); 2.50(m, 2H); 2.34(m, 4H); 1.87(m, 2H); 1.75(m, 2H); 1.66-1.03 |
| | (m, 18H); 1.18(d, 3H) |
| | EI; TSQ 700; source 180° C.; 70V; 200uA: 568(M+.); 537; 485; 402; 167 |
| 70 | ESI POS; AQA; solvent: MeOH/spray 3kV/skimmer: 20V/probe 135° C.: 599 |
| | (MH+) |
| 71 | ¹H NMR(DMSO, 343K)δ: 8.27(d br, 1H); 8.01(d, 1H); 7.85(d, 1H); 7.75(dd, |
| | 1H); 7.61(dd, 1H); 7.56(m, 2H); 7.50-7.39(m, 3H); 3.92(m, 1H); 3.57(s, 2H); 2.50 |
| | (m, 2H); 2.34(m, 4H); 2.14(m, 2H); 1.98(m, 1H); 1.82-1.62(m, 4H); 1.60-0.85(m, |
| | 15H); 0.94(d, 3H); 0.91(d, 3H); 0.89(d, 3H) |
| 72 | ¹H NMR(DMSO, 343K)δ: 8.91(d br, 1H); 8.01(d, 1H); 7.74(dd, 1H); 7.71(d, |
| | 1H); 7.59-7.42(m, 7H); 7.34(m, 1H); 7.21(dd, 1H); 7.16(d, 1H); 5.40(dt, 1H); 3.51 |
| | and 3.45(Abq, 2H); 2.41(m, 2H); 2.32(m, 4H); 2.01-1.76(m, 3H); 1.60(m, 2H); |
| | 1.48-1.30(m, 8H); 1.08(m, 2H); 0.97(t, 3H) |
| 73 | ¹H NMR(DMSO, 343K as a base)δ: 8.82(d br, 1H); 7.53-7.25(m, 11H); 6.90(s, |
| | 1H); 6.19 and 6.12(ABq, 2H); 5.05(dt; 1H); 3.45 and 3.40(Abq, 2H); 2.53-2.36(m, |
| | 8H); 2.07(m, 1H); 1.87(m, 2H); 1.65-1.34(m, 8H); 1.11(m, 2H); 0.95(t, 3H) |
| | ESI POS; AQA; solvent: MeOH/spray 3kV/skimmer: 20V/probe 135° C.: 591 |
| | (MH+) |

TABLE 3
Chemical names of parent compounds of Examples of Table 1 (names generated by
Beilstein's Autonom)

| Ex | Chemical name |
|---|---|
| 1 | 8-[1,4′]Bipiperidinyl-1′-ylmethyl-7-phenyl-2,3-dihydro-[1,4]dioxino[2,3-g]quinoline- |
| | 9-carboxylic acid ((S)-1-cyclohexyl-ethyl)-amide |
| 2 | 7-[1,4′]Bipiperidinyl-1′-ylmethyl-6-phenyl-[1,3]dioxolo[4,5-g]quinoline-9- |
| | carboxylic acid ((S)-1-cyclohexyl-ethyl)-amide |
| 3 | 3-[1,4′]Bipiperidinyl-1′-ylmethyl-4-((S)-1-cyclohexyl-ethylcarbamoyl)-2-phenyl- |
| | quinoline-7-carboxylic acid |
| 4 | 3-[1,4′]Bipiperidinyl-1′-ylmethyl-2-phenyl-quinoline-4-carboxylic acid (thiophen-2- |
| | ylmethyl)-amide |
| 5 | 3-[1,4′]Bipiperidinyl-1′-ylmethyl-2-phenyl-quinoline-4-carboxylic acid 2- |
| | trifluoromethyl-benzylamide |
| 6 | 3-[1,4′]Bipiperidinyl-1′-ylmethyl-2-phenyl-quinoline-4-carboxylic acid 4-chloro- |
| | benzylamide |
| 7 | 3-[1,4′]Bipiperidinyl-1′-ylmethyl-2-phenyl-quinoline-4-carboxylic acid 2-fluoro- |
| | benzylamide |
| 8 | 3-[1,4′]Bipiperidinyl-1′-ylmethyl-2-phenyl-quinoline-4-carboxylic acid (pyridin-2- |
| | ylmethyl)-amide |
| 9 | 3-[1,4′]Bipiperidinyl-1′-ylmethyl-2-phenyl-quinoline-4-carboxylic acid (tetrahydro- |
| | furan-2-ylmethyl)-amide |
| 10 | 3-[1,4′]Bipiperidinyl-1′-ylmethyl-2-phenyl-quinoline-4-carboxylic acid ((1S,2R,5S)- |
| | 6,6-dimethyl-bicyclo[3.1.1]hept-2-ylmethyl)-amide |
| 11 | 3-[1,4′]Bipiperidinyl-1′-ylmethyl-2-phenyl-quinoline-4-carboxylic acid (furan-2- |
| | ylmethyl)-amide |
| 12 | 3-[1,4′]Bipiperidinyl-1′-ylmethyl-2-phenyl-quinoline-4-carboxylic acid (1,1-dioxo- |
| | tetrahydro-1l⁶-thiophen-3-yl)-amide |
| 13 | 3-[1,4′]Bipiperidinyl-1′-ylmethyl-2-phenyl-quinoline-4-carboxylic acid (1,5- |
| | dimethyl-1H-pyrazol-4-ylmethyl)-amide |
| 14 | 2-({[1-(3-[1,4′]Bipiperidinyl-1′-ylmethyl-2-phenyl-quinolin-4-yl)-methanoyl]- |
| | amino}-methyl)-pyrrolidine-1-carboxylic acid tert-butyl ester |
| 15 | 3-[1,4′]Bipiperidinyl-1′-ylmethyl-2-phenyl-quinoline-4-carboxylic acid |
| | cyclohexylmethyl-amide |
| 16 | 3-[1,4′]Bipiperidinyl-1′-ylmethyl-2-phenyl-quinoline-4-carboxylic acid (pyridin-4- |
| | ylmethyl)-amide |
| 17 | 3-[1,4′]Bipiperidinyl-1′-ylmethyl-2-phenyl-quinoline-4-carboxylic acid ((R)-1- |
| | cyclohexyl-ethyl)-amide |
| 18 | 3-[1,4′]Bipiperidinyl-1′-ylmethyl-2-phenyl-quinoline-4-carboxylic acid 2-methoxy- |
| | benzylamide |
| 19 | 3-[1,4′]Bipiperidinyl-1′-ylmethyl-2-phenyl-quinoline-4-carboxylic acid (2,5- |
| | dimethoxy-2,5-dihydro-furan-2-ylmethyl)-amide |
| 20 | 3-[1,4′]Bipiperidinyl-1′-ylmethyl-2-phenyl-quinoline-4-carboxylic acid 4- |
| | dimethylamino-benzylamide |
| 21 | 3-[1,4′]Bipiperidinyl-1′-ylmethyl-2-phenyl-quinoline-4-carboxylic acid (2,2- |
| | dimethyl-[1,3]dioxolan-4-ylmethyl)-amide |
| 22 | 3-[1,4′]Bipiperidinyl-1′-ylmethyl-2-phenyl-quinoline-4-carboxylic acid 3,4-dichloro- |
| | benzylamide |
| 23 | [3-({[1-(3-[1,4′]Bipiperidinyl-1′-ylmethyl-2-phenyl-quinolin-4-yl)-methanoyl]- |
| | amino}-methyl)-phenyl]-carbamic acid tert-butyl ester |
| 24 | 3-({[1-(3-[1,4′]Bipiperidinyl-1′-ylmethyl-2-phenyl-quinolin-4-yl)-methanoyl]- |
| | amino}-methyl)-piperidine-1-carboxylic acid tert-butyl ester |
| 25 | 3-[1,4′]Bipiperidinyl-1′-ylmethyl-2-phenyl-quinoline-4-carboxylic acid 4- |
| | methanesulfonyl-benzylamide |
| 26 | 3-[1,4′]Bipiperidinyl-1′-ylmethyl-2-phenyl-quinoline-4-carboxylic acid |
| | (benzo[1,3]dioxol-5-ylmethyl)-amide |
| 27 | 3-[1,4′]Bipiperidinyl-1′-ylmethyl-2-phenyl-quinoline-4-carboxylic acid 3,5-dimethyl- |
| | benzylamide |
| 28 | 3-[1,4′]Bipiperidinyl-1′-ylmethyl-2-phenyl-quinoline-4-carboxylic acid 4-tert-butyl- |
| | benzylamide |
| 29 | 3-[1,4′]Bipiperidinyl-1′-ylmethyl-2-phenyl-quinoline-4-carboxylic acid 3-chloro- |
| | benzylamide |
| 30 | 3-[1,4′]Bipiperidinyl-1′-ylmethyl-2-phenyl-quinoline-4-carboxylic acid 3-bromo- |
| | benzylamide |
| 31 | 3-[1,4′]Bipiperidinyl-1′-ylmethyl-2-phenyl-quinoline-4-carboxylic acid 4-methyl- |
| | benzylamide |
| 32 | 3-[1,4′]Bipiperidinyl-1′-ylmethyl-2-phenyl-quinoline-4-carboxylic acid 2,3,6- |
| | trifluoro-benzylamide |
| 33 | 3-[1,4′]Bipiperidinyl-1′-ylmethyl-2-phenyl-quinoline-4-carboxylic acid (biphenyl-3- |
| | ylmethyl)-amide |
| 34 | 3-[1,4′]Bipiperidinyl-1′-ylmethyl-2-phenyl-quinoline-4-carboxylic acid (1H-indol-3- |
| | ylmethyl)-amide |
| 35 | [3-({[1-(3-[1,4′]Bipiperidinyl-1′-ylmethyl-2-phenyl-quinolin-4-yl)-methanoyl]- |
| | amino}-methyl)-benzyl]-carbamic acid tert-butyl ester |
| 36 | 3-[1,4′]Bipiperidinyl-1′-ylmethyl-2-phenyl-quinoline-4-carboxylic acid (1,1-diphenyl- |
| | methyl)-amide |
| 37 | 3-[1,4′]Bipiperidinyl-1′-ylmethyl-2-phenyl-quinoline-4-carboxylic acid [(S)-1-(4- |
| | methoxy-phenyl)-ethyl]-amide |
| 38 | 3-[1,4′]Bipiperidinyl-1′-ylmethyl-2-phenyl-quinoline-4-carboxylic acid [(S)-1-(3- |
| | methoxy-phenyl)-ethyl]-amide |
| 39 | 3-[1,4′]Bipiperidinyl-1′-ylmethyl-2-phenyl-quinoline-4-carboxylic acid (thiophen-3- |
| | ylmethyl)-amide |
| 40 | 3-[1,4′]Bipiperidinyl-1′-ylmethyl-2-phenyl-quinoline-4-carboxylic acid (1-thiophen- |
| | 2-yl-ethyl)-amide |
| 41 | 3-[1,4′]Bipiperidinyl-1′-ylmethyl-2-phenyl-quinoline-4-carboxylic acid [1-(2-methyl- |
| | thiazol-4-yl)-ethyl]-amide |
| 42 | 3-[1,4′]Bipiperidinyl-1′-ylmethyl-2-phenyl-quinoline-4-carboxylic acid (4-methyl- |
| | thiazol-2-ylmethyl)-amide |
| 43 | 3-[1,4′]Bipiperidinyl-1′-ylmethyl-2-phenyl-quinoline-4-carboxylic acid [1-(4-methyl- |
| | thiazol-2-yl) -ethyl]-amide |
| 44 | 3-[1,4′]Bipiperidinyl-1′-ylmethyl-2-phenyl-quinoline-4-carboxylic acid |
| | (benzothiazol-2-ylmethyl)-amide |
| 45 | 3-[1,4′]Bipiperidinyl-1′-ylmethyl-2-phenyl-quinoline-4-carboxylic acid (5-methyl- |
| | [1,3,4]oxadiazol-2-ylmethyl)-amide |
| 46 | 3-[1,4′]Bipiperidinyl-1′-ylmethyl-2-phenyl-quinoline-4-carboxylic acid (3-methyl- |
| | isoxazol-5-ylmethyl)-amide |
| 47 | 3-[1,4′]Bipiperidinyl-1′-ylmethyl-2-phenyl-quinoline-4-carboxylic acid 4-fluoro- |
| | benzylamide |
| 48 | 3-[1,4′]Bipiperidinyl-1′-ylmethyl-2-phenyl-quinoline-4-carboxylic acid (2-phenyl- |
| | 2H-[1,2,3]triazol-4-ylmethyl)-amide |
| 49 | 3-[1,4′]Bipiperidinyl-1′-ylmethyl-2-phenyl-quinoline-4-carboxylic acid [(1S,9aR)-1- |
| | (octahydro-quinolizin-1-yl)methyl]-amide |
| 50 | 3-[1,4′]Bipiperidinyl-1′-ylmethyl-2-phenyl-quinoline-4-carboxylic acid [1-(3,5- |
| | dimethyl-1H-pyrazol-4-yl)-ethyl]-amide |
| 51 | 3-[1,4′]Bipiperidinyl-1′-ylmethyl-2-phenyl-quinoline-4-carboxylic acid (1,3- |
| | dimethyl-1H-pyrazol-4-yl)-ethyl]-amide |
| 52 | 3-[1,4′]Bipiperidinyl-1′-ylmethyl-2-phenyl-quinoline-4-carboxylic acid (1,3,5- |
| | trimethyl-1H-pyrazol-4-ylmethyl)-amide |
| 53 | 3-[1,4′]Bipiperidinyl-1′-ylmethyl-2-phenyl-quinoline-4-carboxylic acid (1-methyl- |
| | 1H-pyrazol-4-ylmethyl)-amide |
| 54 | 3-[1,4′]Bipiperidinyl-1′-ylmethyl-2-phenyl-quinoline-4-carboxylic acid (2,2- |
| | dimethyl-tetrahydro-pyran-4-yl)-amide |
| 55 | 3-[1,4′]Bipiperidinyl-1′-ylmethyl-2-phenyl-quinoline-4-carboxylic acid (2-oxo- |
| | azepan-3-yl)-amide |
| 56 | 3-[1,4′]Bipiperidinyl-1′-ylmethyl-2-phenyl-quinoline-4-carboxylic acid [1-(2,4- |
| | dimethyl-phenyl)-ethyl]-amide |
| 57 | 3-[1,4′]Bipiperidinyl-1′-ylmethyl-2-phenyl-quinoline-4-carboxylic acid (5-methyl- |
| | furan-2-ylmethyl)-amide |
| 58 | 3-[1,4′]Bipiperidinyl-1′-ylmethyl-2-phenyl-quinoline-4-carboxylic acid [2-(4- |
| | methoxy-phenyl)-[1,3]dioxolan-2-ylmethyl]-amide |
| 59 | 3-[1,4′]Bipiperidinyl-1′-ylmethyl-2-phenyl-quinoline-4-carboxylic acid 4-isopropyl- |
| | benzylamide |
| 60 | 3-[1,4′]Bipiperidinyl-1′-ylmethyl-2-phenyl-quinoline-4-carboxylic acid (pyridin-3- |
| | ylmethyl)-amide |
| 61 | 3-[1,4′]Bipiperidinyl-1′-ylmethyl-2-phenyl-quinoline-4-carboxylic acid 3-fluoro- |
| | benzylamide |
| 62 | 3-[1,4′]Bipiperidinyl-1′-ylmethyl-2-phenyl-quinoline-4-carboxylic acid 3- |
| | trifluoromethyl-benzylamide |
| 63 | 3-[1,4′]Bipiperidinyl-1′-ylmethyl-2-phenyl-quinoline-4-carboxylic acid 3-methoxy- |
| | benzylamide |
| 64 | 3-[1,4′]Bipiperidinyl-1′-ylmethyl-2-phenyl-quinoline-4-carboxylic acid 4-methoxy- |
| | benzylamide |
| 65 | 3-[1,4′]Bipiperidinyl-1′-ylmethyl-2-phenyl-quinoline-4-carboxylic acid 4- |
| | trifluoromethyl-benzylamide |
| 66 | 3-[1,4′]Bipiperidinyl-1′-ylmethyl-2-phenyl-quinoline-4-carboxylic acid (1-methyl-1- |
| | phenyl-ethyl)-amide |
| 67 | 3-[1,4′]Bipiperidinyl-1′-ylmethyl-2-phenyl-quinoline-4-carboxylic acid (1- |
| | cyclohexyl-1-methyl-ethyl)-amide |
| 68 | 3-(3-[1,4′]Bipiperidinyl-1′-yl-propyl)-2-phenyl-quinoline-4-carboxylic acid ((S)-1- |
| | cyclohexyl-ethyl)-amide |
| 69 | 3-[1,4′]Bipiperidinyl-1′-ylmethyl-2-phenyl-quinoline-4-carboxylic acid [1-(4- |
| | methoxy-cyclohexyl)-ethyl]-amide |
| 70 | 3-[1,4′]Bipiperidinyl-1′-ylmethyl-6,7-dimethoxy-2-phenyl-quinoline-4-carboxylic |
| | acid ((S)-1-cyclohexyl-ethyl)-amide |
| 71 | 3-[1,4′]Bipiperidinyl-1′-ylmethyl-2-phenyl-quinoline-4-carboxylic acid (2-isopropyl- |
| | 5-methyl-cyclohexyl)-amide |
| 72 | 3-[1,4′]Bipiperidinyl-1′-ylmethyl-2-phenyl-quinoline-4-carboxylic acid [1-(2-fluoro- |
| | phenyl)-propyl]-amide |
| 73 | 7-[1,4′]Bipiperidinyl-1′-ylmethyl-6-phenyl-[1,3]dioxolo[4,5-g]quinoline-8-carboxylic |
| | acid ((S)-1-phenyl-propyl)-amide |

1 A compound of formula (I) below or a pharmaceutically acceptable salt or solvate thereof: 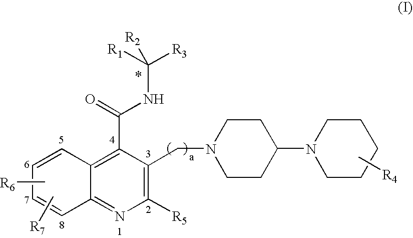
wherein:
R₁ is H or alkyl, R₂ is R₈R₉, and R₃ is H, alkyl or cycloalkyl, optionally substituted by one or more fluorines; or R₂ is R₈R₉ and R₁ and R₃ together with the carbon atom to which they are attached form a cycloalkyl, aryl or heterocyclic ring having 4-7 ring members, which ring R₁/R₃ is unsubstituted or is substituted one or more times by one or more of oxo, hydroxy, halogen, nitro, cyano, carboxy, and amino; or R₃ is H and and R₁ and R₂ together with the carbon atom to which they are attached form a 47 membered cycloalkyl, aryl or heterocyclic ring, which cycloalkyl, aryl or heterocyclic ring R₁/R₂ is unsubstituted or is substituted one or more times by one or more substituents selected from alkyl, halo, hydroxy, amino, cyano, nitro, carboxy and oxo, and/or is fused with a cycloalkyl, aryl or 4-7-membered heterocyclic ring;
R₈ represents a single bond or alkyl, optionally substituted by one or more fluorines; R₉ represents an aryl ring or a cycloalkyl or heterocyclic ring having 3-10 ring members, which aryl, cycloalkyl or heterocyclic ring R₉ is unsubstituted or is substituted by R₁₀, which aryl, cycloalkyl or heterocyclic ring R₉ is optionally fused with an aryl, cycloalkyl or 4-7-membered heterocyclic ring;
R₁₀ represents one or more ring substituents independently selected from oxo, hydroxy, halogen, nitro, cyano, carboxy, amino; and/or branched or linear alkyl, alkenyl, alkoxy, or aryl, or a hydroxylated derivative thereof; and/or a branched or linear C₁₋₆ alkyl chain, optionally including one or more of amino, amido, ether, ester, carboxy, sulfonyl, alkenyl, alkynyl, cycloalkyl or aryl functionality and optionally substituted one or more times by one or more of oxo, hydroxy, halogen, nitro, cyano, carboxy, and amino; and/or R₁₀ represents a bridging moiety which is arranged to bridge two ring members in said aryl, cycloalkyl or heterocyclic ring, which bridging moiety comprises mono- or di-oxyalkylene or alkyl;
R₄ represents H or one or more fluorine substituents;
R₅ is branched or linear alkyl, cycloalkyl, cycloalkylalkyl, aryl, or a single or fused ring aromatic heterocyclic group;
R₆ represents H or up to three substituents independently selected from the list consisting of: alkyl, alkenyl, aryl, alkoxy or a hydroxylated derivative thereof, hydroxy, halogen, nitro, cyano, carboxy, alkylcarboxy, alkylcarboxyalkyl, trifluoromethyl, amino or mono- or di- alkylamino; or R₆ represents a bridging moiety which is arranged to bridge two adjacent ring atoms, which bridging moiety comprises alkyl or dioxyalkylene;
R₇ is H, alkoxy or halo;
a is 1-6; and
R₂ or R₅ may optionally be substituted one or more times by halo, hydroxy, amino, cyano, nitro, carboxy or oxo;
not being a compound in which R₄ is H, R₅ is unsubstituted phenyl, R₇ is H, a is 1, and R₁, R₂, R₃ and R₆ are selected from the following:

| 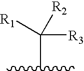 | R₆ |
|---|---|
| 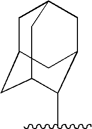 | H |
| 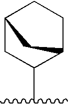 | H |
| 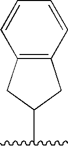 | H |
| 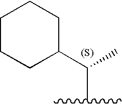 | H |
| 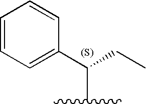 | H |
| 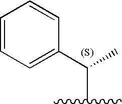 | H |
| 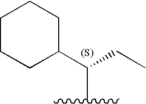 | H |
| 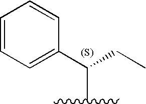 | 7-OMe, 8-Br |
| 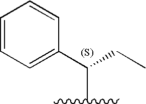 | 7-OMe |
| 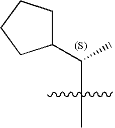 | H |
| 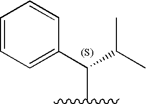 | H |
| 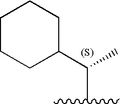 | 7-OMe |
| 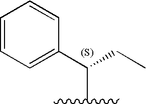 | 7-OH, 8-Cl |
| 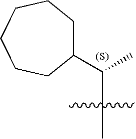 | H |
| 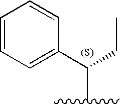 | 7-OH |
| 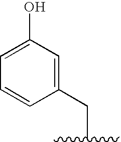 | H |
| 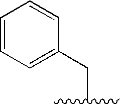 | H |
| 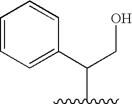 | H |
| 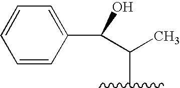 | H |
| 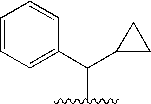 | H |
| 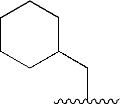 | H |
| 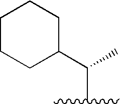 | 6-OH, 7-OH |
| 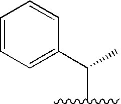 | 6-OH, 7-OH |
| 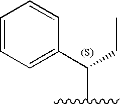 | 6-OEtOH, 7-OEtOH |
| 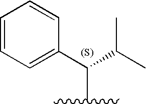 | 6-OH, 7-OH |
| 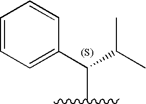 | 6-OMe, 7-OMe |
| 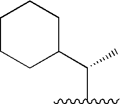 | 6-Cl, 7-Cl, 7-F, 8-F |
| 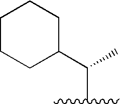 | 6-CF₃, 7-CF₃ |

with the further proviso that said compound of formula (I) is not a compound selected from the following:
3-[1,4′]Bipiperidinyl-1′-ylmethyl-2-thiophen-2-yl-quinoline-4-carboxylic acid ((S)-1-cyclohexyl-ethyl)-amide;
3-[1,4′]Bipiperidinyl-1′-ylmethyl-2-(4-fluoro-phenyl)-quinoline-4-carboxylic acid ((S)-1-cyclohexyl-ethyl)-amide;
3-[1,4′]Bipiperidinyl-1′-ylmethyl-2-(4-trifluoromethyl-phenyl)-quinoline-4-carboxylic acid ((S)-1-cyclohexyl-ethyl)-amide; and
3-[1,4′]Bipiperidinyl-1′-ylmethyl-2-(2-fluoro-phenyl)-quinoline-4-carboxylic acid ((S)-1-cyclohexyl-ethyl)amide.

2 A compound as claimed in claim 1, wherein R₂ is R₈R₉ and R₈ represents a single bond or methyl.

3 A compound as claimed in claim 1 or claim 2, wherein R₂ is R₈R₉ and R₉ represents phenyl, or cyclohexyl, or a saturated or unsaturated heterocyclic ring having 5 or 6 ring members and including one or more heteroatoms selected from N, O and S.

4 A compound as claimed in any of claims 1-3, wherein R₂ is R₈R₉, R₉ is substituted by R₁₀, and R₁₀ includes 1-3 ring substituents selected from bromo, chloro, fluoro, methyl, ethyl, methoxy, ethoxy, phenyl and cyclohexyl, each of which substituents may optionally be substituted one or more times by halo such as fluoro.

5 A compound as claimed in claim 4, wherein R₂ is R₈R₉, R₉ is substituted by R₁₀, and R₁₀ includes one ring substituent which is trifluoromethyl.

6 A compound as claimed in any of claims 1-5, wherein R₂ is R₈R₉, R₉ is substituted by R₁₀, and R₁₀ includes one ring substituent which is branched or linear alkoxy, alkylcarboxy, alkylamino, alkylsulfonyl, alkylether, or alkyloxyamido, which ring substituent is linked to R₉ by a single bond or by C₁₋₃ alkyl.

7 A compound as claimed in any preceding claim, wherein R₂ is R₈R₉, R₉ is substituted by R₁₀, and R₁₀ includes one ring substituent which is a bridging moiety comprising ethyl or dioxyethylene.

8 A compound as claimed in any preceding claim, wherein R₂ is R₈R₉, and R₉ is an aryl, cycloalkyl or 3-10-membered heterocyclic ring which is fused to a phenyl or cyclohexyl ring.

9 A compound as claimed in any preceding claim, wherein R₂ is R₈R₉ and R₁ and R₃ together with the carbon atom to which they are attached form a 5- or 6-membered heterocyclic ring R₁/R₃ comprising one or more heteroatoms selected from N, O and S.

10 A compound as claimed in claim 9, wherein said heterocyclic ring R₁/R₃ comprises five ring members including two O heteroatoms.

11 A compound as claimed in any of claims 1-8, wherein R₂ is R₈R₉ and R₃ is methyl, ethyl, iso-propyl or phenyl.

12 A compound as claimed in claim 11, wherein R₁ is H or methyl.

13 A compound as claimed in claim 1, wherein R₃ is H and R₁ and R₂ together with the carbon atom to which they are attached form a 5-7 membered heterocyclic ring R₁/R₂ comprising one heteroatom selected from N, O and S.

14 A compound as claimed in claim 13, wherein said heterocyclic ring R₁/R₂ is substituted one or more times by one or more substituents selected from oxo, methyl and ethyl.

15 A compound as claimed in any preceding claim, wherein R₅ is unsubstituted phenyl.

16 A compound as claimed in any preceding claim, wherein R₆ represents hydrogen, chloro or bromo.

17 A compound as claimed in any of claims 1-15, wherein R₆ represents one ring substituent, which is hydroxy, methoxy, ethoxy or a hydroxy-terminated derivative of methoxy or ethoxy, or carboxy or methylcarboxy or ethylcarboxy.

18 A compound as claimed in claim 17, wherein said one ring substituent is located at the 6 or 7 position around said ring.

19 A compound as claimed in any of claims 1-15, wherein R₆ represents a bridging moiety which is arranged to bridge two adjacent ring atoms, which bridging moiety comprises dioxymethylene or dioxyethylene.

20 A Compound as claimed in claim 19, wherein said bridging moiety is arranged to bridge the 6 and 7 positions around said ring.

21 A compound as claimed in any preceding claim, wherein R₇ represents hydrogen.

22 A compound as claimed in any preceding claim, wherein a is 1, 2 or 3.

23 A compound as claimed in any preceding claim, wherein a is 1.

24 A compound as claimed in any preceding claim, wherein R₄ is H.

25 A compound as claimed in any preceding claim, wherein a is 1, R₁ is H, R₃ is H, R₄ is H, R₅ is unsubstituted phenyl, R₆ is H, R₇ is H, and R₂ is one of the following:

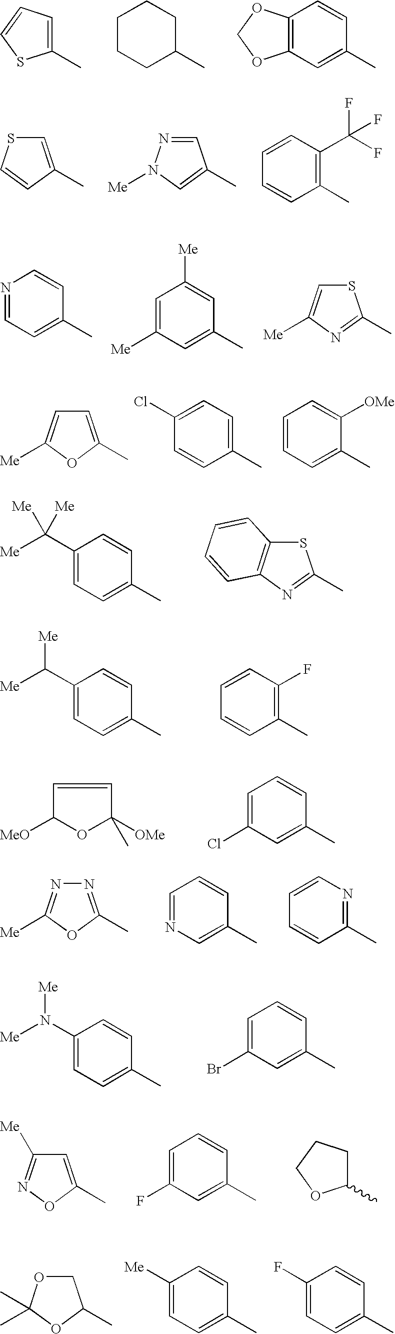

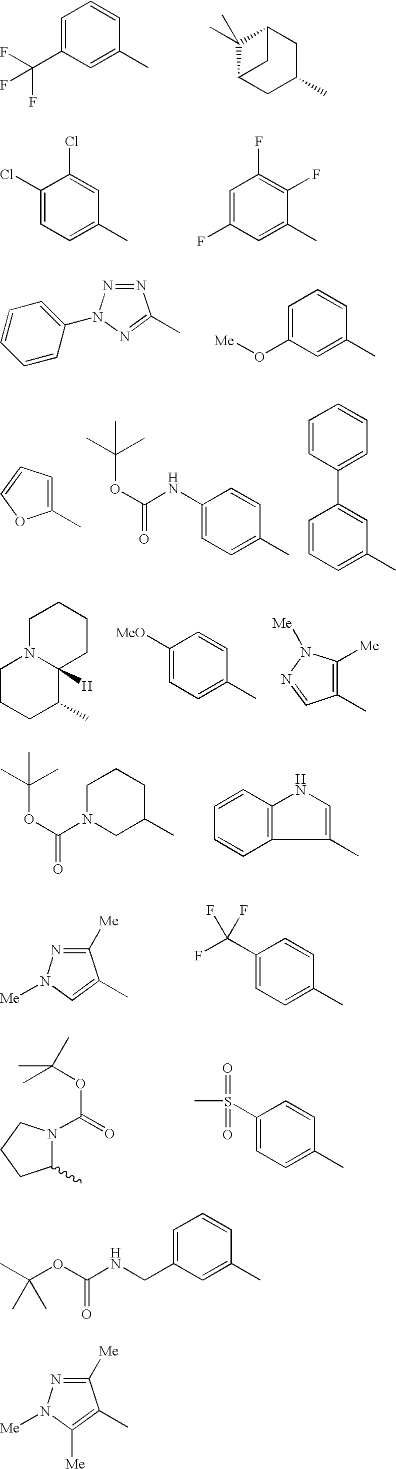

26 A compound as claimed in any of claims 1-24, wherein a is 1, R₄ is H, R₅ is unsubstituted phenyl, R₆ is H, R₇ is H, and R₁, R₂ and R₃ are selected from the following:

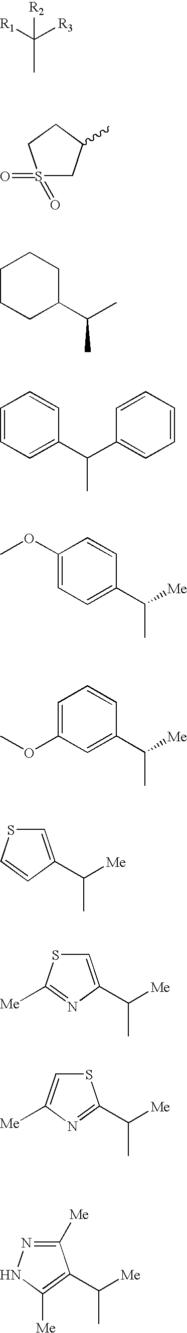

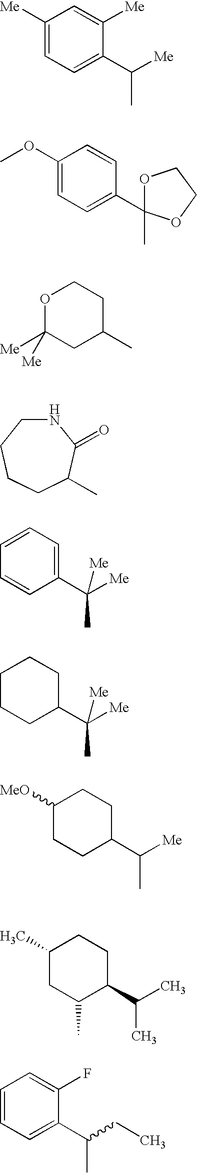

27 A compound as claimed in any of claims 1-24, selected from the following:

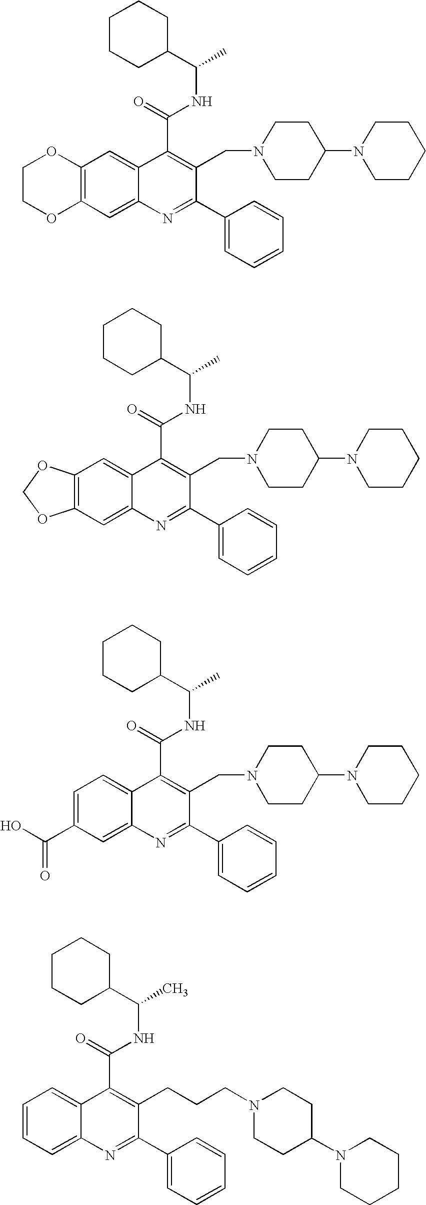

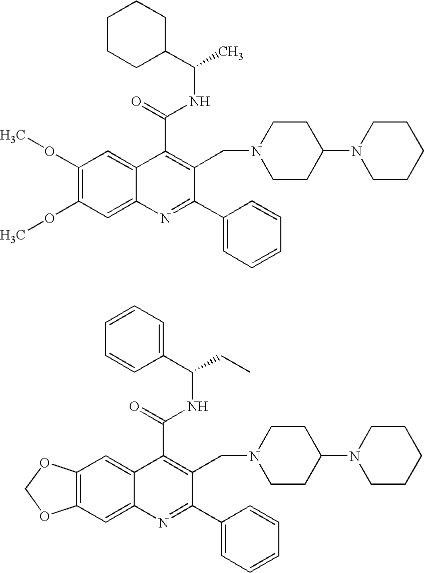

28 A process for the preparation of a compound of formula (I) according to any of claims 1-27, or a salt thereof and/or a solvate thereof, which process comprises reacting a compound of formula (II) or an active derivative thereof: 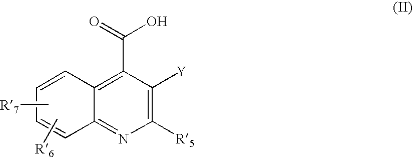
wherein R′₅, R′₆, and R′₇ are R₅, R₆, and R₇ respectively as defined in relation to formula (I) as claimed in claim 1 or a group convertible to R₅, R₆, and R₇ respectively, and Y′ is a group of formula (Y) or a group convertible thereto 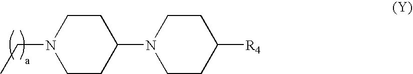
where R₄ is defined as in relation to formula (I) as claimed in claim 1, with a compound of formula (III): 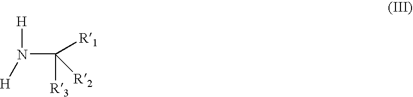
wherein R′₁, R′₂ and R′₃ are R₁, R₂ and R₃ as defined for formula (I) as claimed in claim 1 or a group or atom convertible to R₁, R₂ and R₃ respectively; to form a compound of formula (Ib): 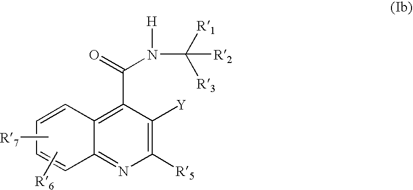
wherein R′₁, R′₂, R′₃, R′₅, R′₆, R′₇ and Y′ are as defined in claim 1, and thereafter carrying out one or more of the following optional steps:
(i) converting any one of R′₁, R′₂, R′₃, R′₅, R′₆, R′₇ and Y′ to R₁, R₂, R₃, R₅, R₆, R₇ and Y respectively as required, to obtain a compound of formula (I) as claimed in claim 1;
(ii) converting a compound of formula (I) as claimed in claim 1 into another compound of formula (I) as claimed in claim 1; and
(iii) preparing a salt of the compound of formula (I) as claimed in claim 1 and/or a solvate thereof.

29 A pharmaceutical composition comprising a compound of formula (I) according to any of claims 1-27, or a pharmaceutically acceptable salt or solvate thereof, and a pharmaceutically acceptable carrier.

30 A compound of formula (I) according to any of claims 1-27, or a pharmaceutically acceptable salt or solvate thereof, for use as an active therapeutic substance.

31 A compound of formula (I) according to any of claims 1-27, or a pharmaceutically acceptable salt or solvate thereof, for the treatment or prophylaxis of the Primary and Secondary Conditions.

32 Use of a compound of formula (I) according to any of claims 1-27, or a pharmaceutically acceptable salt or solvate thereof, in the manufacture of a medicament for the treatment of the Primary and Secondary Conditions.

33 A method for the treatment and/or prophylaxis of the Primary and Secondary Conditions in mammals, particularly humans, which comprises administering to the mammal in need of such treatment and/or prophylaxis an effective, non-toxic pharmaceutically acceptable amount of a compound of formula (I) according to any of claims 1-27 or a pharmaceutically acceptable salt or solvate thereof.
